(19) United States
(12) Patent Application Publication
(10) Pub. No.: US 2024/0189365 A1
(43) Pub. Date: Jun. 13, 2024

(54) Glycan Targets of Serum IgG and IgM Antibodies

(71) Applicant: REGENTS OF THE UNIVERSITY OF MINNESOTA, Minneapolis, MN (US)

(72) Inventor: Christopher BURLAK, Minneapolis, MN (US)

(73) Assignee: REGENTS OF THE UNIVERSITY OF MINNESOTA, Minneapolis, MN (US)

(21) Appl. No.: 18/286,955

(22) PCT Filed: Apr. 14, 2022
(86) PCT No.: PCT/US2022/024859

§ 371 Date: Oct. 13, 2023

Related U.S. Application Data
(60) Provisional application No. 63/175,273, filed on Apr. 15, 2021.

(51) Int. Cl. A61K 35/407; A61K 35/22; A61K 35/34; A61K 35/36; A61K 35/38; A61K 35/39; A61K 35/42; A61K 35/55; A61P 37/06; C12N 5/071

(52) U.S. Cl. CPC A61K 35/407; A61K 35/22; A61K 35/34; A61K 35/36; A61K 35/38; A61K 35/39; A61K 35/42; A61K 35/55; A61P 37/06; C12N 5/0676; C12N 2510/00

(57) ABSTRACT

Provided herein are genetically modified porcine cells comprising a reduced or eliminated level of β1,3-N-acetylglucosaminyltransferase 5 (B3gnt5) activity as compared to a normal porcine cell, and methods of improving a rejection related symptom by transplanting porcine cells, tissues or organs to a subject in need thereof.

### RELATED APPLICATION

This application claims priority to U.S. Provisional Application No. 63/175,273 that was filed on Apr. 15, 2021. The entire content of the application referenced above IS hereby incorporated by reference.

### BACKGROUND

For some clinical disorders and diseases, therapies include transplantation of human cells, tissues, and organs (i.e., allotransplant). The demand for donor material, however, largely outstrips the available supply. On average, 20 people die each day in the United States waiting for an organ transplant, and over 112,000 people are waiting for transplants. One solution to increase the number of donor organs is to use organs from a different species, called xenotransplantation. Porcine organs are a potential organ donor pool for humans, as many organs have anatomical and physiological characteristics that are similar to humans. Due to genetic differences between the two species, however, xenogeneic transplants trigger immune responses in the recipient, leading to organ rejection and failure. For instance, following xenotransplantation, host macrophages are rapidly recruited to the xenograft and quickly phagocytize the transplanted porcine cells, leading to rejection of the xenograft. Consequently, there are immunological barriers that must be overcome for successful xenotransplantation. Accordingly, there remains a need in the art for xenogeneic donor cells, tissues, and organs at any stage of development that are better able to evade the immune response triggered in the recipient, allowing for longer xenograft survival.

### SUMMARY

In an aspect, provided herein is a genetically modified porcine cell comprising a reduced level of β1,3-N-acetylglucosaminyltransferase 5 activity as compared to a normal porcine cell.

In an aspect, provided herein is a genetically modified porcine cell comprising a biologically inactive or deleted β1,3-N-acetylglucosaminyltransferase 5 (B3gnt5) gene.

In an aspect, provided herein is a genetically modified porcine cell, wherein the genetic modification comprises a modification to the genome of the porcine cell that results in the lack of expression of functional β1,3-N-acetylglucosaminyltransferase 5, wherein the genetically modified porcine cell exhibits reduced binding to human immunoglobulins relative to a porcine cell lacking the genetic modifications.

In an aspect, provided herein is a genetically modified porcine cell comprising a reduced level of Lacto-N-neotetraose (LNnT) glycan (Galb1-4GlcNAcb1-3Galb1) on its cell surface as compared to a normal porcine cell.

In an aspect, provided herein is a porcine organ or tissue comprising the genetically modified porcine cell described above.

In an aspect, provided herein is a method of treating a subject in need of an organ transplant, the method comprising administering a therapeutically sufficient amount of the genetically modified porcine cell, tissue or organ described above to the subject.

In an aspect, provided herein is a method of improving a rejection related symptom in a human subject comprising transplanting porcine transplant material having reduced levels of β1,3-N-acetylglucosaminyltransferase 5 activity into a human subject in need of a transplant, wherein a rejection related symptom is improved as compared to when porcine transplant material from a wild-type pig is transplanted into a human subject.

In an aspect, provided herein is a method of improving a rejection related symptom in a human subject comprising transplanting porcine transplant material having reduced levels of Lacto-N-neotetraose (LNnT) glycan (Galb1-4GlcNAcb1-3Galb1) on cell surfaces into a human subject in need of a transplant, wherein a rejection related symptom is improved as compared to when porcine transplant material from a wild-type pig is transplanted into a human subject.

In an aspect, provided herein is a method of improving a rejection related symptom in a human subject comprising transplanting porcine transplant material having reduced levels of Lacto-N-neotetraose (LNnT) epitope (Galb1-4GlcNAcb1-3Galb1) into a human subject in need of a transplant, wherein a rejection related symptom is improved as compared to when porcine transplant material from a wild-type pig is transplanted into a human subject.

### BRIEF DESCRIPTION OF DRAWINGS

FIGS. 1A-1B. The distribution (log-transformed base 2) of 50 highest serum IgM (FIG. 1A) and IgG (FIG. 1B) elicited antibody signals in five monkeys. The number after the abbreviation indicates the average number of carbohydrates per molecule of albumin. The circle represents the median of the five monkeys, and the bars represent the standard deviation for the group.

FIG. 2. Comparison of antibody signals in each monkey. Signals for each carbohydrate in posttransplant serum were plotted against pretransplant naïve serum. Signals are on log 2 scale. The plots in the larger outer triangles represent carbohydrate antigens that are increased in the posttransplant serum. The plots in the smaller inner triangles represent carbohydrate antigen that are significantly increased (the changes of normalized signal intensity ≥1.5) in the posttransplant serum. Blood type of a monkey is noted in brackets.

FIGS. 3A-3B. The list of carbohydrates that were significantly elicited in the posttransplant sera (the changes of normalized signal intensity ≥1.5; naïve sera, gray; posttransplant sera, black). The number after the abbreviation indicates the average number of carbohydrates per molecule of albumin. Blood type of a monkey is noted in brackets.

FIG. 4. The list of antibody signals against historically reported α-Gal and non-α-Gal carbohydrate antigens in IgM and IgG repertoires. The α-Gal antigens include carbohydrate structures with Gala1-3Gal epitope. The non-α-Gal antigens include carbohydrate structures with Sd^{a} and Neu5Gc epitopes. The number after the abbreviation indicates the average number of carbohydrates per molecule of albumin. An antibody signal that is significantly increased (the changes of normalized signal intensity ≥1.5) in the posttransplant serum is marked with an asterisk.

FIG. 5. Carbohydrate antigens elicited in common among sensitized NHP sera.

FIGS. 6A-6R. List of serum IgM and IgG natural antibody signals from seven cynomolgas macaques to 408 array components.

FIGS. 7A-7B. Fibroblasts cell transfected with Cas12 and guide RNA for GGTA1, CMAH, B4GalNT2, and B3GNT5. FIG. 7A. Cell sorting for Neu5GC negative cells at 7 days after transfection. Genetics in gates are confirmed by Sanger sequencing. FIG. 7B. Cell sorting for monkey serum IgM low cells at 14 days after transfection.

FIG. 8. Crossmatch for B3GNT5^{-/-} deficient cells using Rhesus serum. Left peak in each graph: non-serum control; middle peak in each graph: clone cells; and right peak in each graph: non-transfected control.

FIG. 9. CDC assay for B3GNT5^{-/-} deficient cells following Rhesus serum treatment.

### DETAILED DESCRIPTION

There is a tremendous shortage of human cells, tissues, and organs to save those in need life-saving therapies. Xenotransplantation could overcome this shortage through pigs genetically modified (i.e., transgenic, genetically engineered) to be organ donors. Limitaitons to this process have included the presence of surface glycans on the cells in pigs that are naturally deleted in humans. These glycans are bound by recipient antibody that fix complement and activate immune cells leading to donor cell death. Deletion of the genes that catalyze these cell surface glycans prevents the formation of the surface glycans making the cells surface immunologically more like the human or primates. The genetically engineered pigs with antigenically reduced phenotypes give rise to cells, tissues, and organs that can be used in a myriad of transplantation therapeutics. The genetically modified animals, organs, tissues, and cells provided herein are based at least in part on the inventor's identification of targets of serum IgG and IgM that are glycans created by unique enzymes that are encoded by primate genes.

The glycan microarray discussed herein probed with monkey serum revealed several targets of serum IgG and IgM that are glycans created by unique enzymes that are encoded by genes. Specifically, the LNnT glycan is created by the enzyme β1,3-N-acetylglucosaminyltransferase 5 gene (B3gnt5). Deletion of this gene through gene specific mutation disrupts the production of the LNnt glycan on the cell surface and prevents antibody binding.

Accordingly, in an aspect, provided herein is a genetically modified porcine cell comprising a reduced level of β1,3-N-acetylglucosaminyltransferase 5 activity as compared to a normal porcine cell.

In an aspect, provided herein is a genetically modified porcine cell comprising a biologically inactive or deleted β1,3-N-acetylglucosaminyltransferase 5 (B3gnt5) gene.

In an aspect, provided herein is a genetically modified porcine cell, wherein the genetic modification comprises a modification to the genome of the porcine cell that results in the lack of expression of functional β1,3-N-acetylglucosaminyltransferase 5, wherein the genetically modified porcine cell exhibits reduced binding to human immunoglobulins relative to a porcine cell lacking the genetic modifications.

In an aspect, provided herein is a genetically modified porcine cell comprising a reduced level of Lacto-N-neotetraose (LNnT) glycan (Galb1-4GlcNAcb1-3Galb1) on its cell surface as compared to a normal porcine cell.

In an aspect provided herein, the genetically modified porcine cell further comprises a reduced level of GlcNAc-Man3 (Mana1-6(GlcNAcb1-2Mana1-3)Manb1-4GlcNAcb) antigen.

In an aspect provided herein, the genetically modified porcine cell further comprises a reduced level of Sd^{a} antigen.

In an aspect provided herein, the porcine cell is selected from skin, heart, liver, kidneys, lung, pancreas, thyroid, and small bowel, or portions thereof.

In an aspect, provided herein is a porcine organ or tissue comprising the genetically modified porcine cell as described herein.

In an aspect provided herein, the porcine organ or tissue of claim 10, wherein the porcine organ or tissue is selected from skin, heart, liver, kidneys, lung, pancreas, thyroid, and small bowel, or portions thereof.

In an aspect, provided herein is a method of treating a subject in need of an organ transplant, the method comprising administering a therapeutically sufficient amount of the genetically modified porcine cell, tissue or organ as described herein to the subject.

In an aspect, provided herein is a method of improving a rejection related symptom in a human subject comprising transplanting porcine transplant material having reduced levels of β1,3-N-acetylglucosaminyltransferase 5 activity into a human subject in need of a transplant, wherein a rejection related symptom is improved as compared to when porcine transplant material from a wild-type pig is transplanted into a human subject.

In an aspect, provided herein is a method of improving a rejection related symptom in a human subject comprising transplanting porcine transplant material having reduced levels of Lacto-N-neotetraose (LNnT) glycan (Galb1-4GlcNAcb1-3Galb1) on cell surfaces into a human subject in need of a transplant, wherein a rejection related symptom is improved as compared to when porcine transplant material from a wild-type pig is transplanted into a human subject.

In an aspect, provided herein is a method of improving a rejection related symptom in a human subject comprising transplanting porcine transplant material having reduced levels of Lacto-N-neotetraose (LNnT) epitope (Galb1-4GlcNAcb1-3Galb1) into a human subject in need of a transplant, wherein a rejection related symptom is improved as compared to when porcine transplant material from a wild-type pig is transplanted into a human subject.

In an aspect provided herein, the rejection related symptom is selected from a cellular rejection response related symptom, a humoral rejection response related symptom, a hyperacute rejection related symptom, an acute humoral xenograft reaction rejection related symptom, and an acute vascular rejection response related symptom.

In certain embodiments, porcine cells are genetically modified to lack expression of functional β1,3-N-acetylglucosaminyltransferase 5 gene (B3gnt5) and/or the the LNnT glycan are further genetically modified to lack expression of other xenoreactive antigens. For example, it will be advantageous in some cases to generate a double, triple, quadruple, or quintuple knockout transgenic pig in which its genome has been genetically modified to mutate or knock out the genes encoding α-Gal antigen and/or Sd^{a} antigen and pCD81 (e.g., α-Gal/Sd^{a} double knock-out). [are there other specific antigens that should be mentioned?] The enzyme α-1,3-galactosyltransferase-1 (GGTA1) is required for synthesis of α-Gal antigen.

As used herein, the term “genetic modification” and its grammatical equivalents can refer to one or more alterations of a nucleic acid, e.g., the nucleic acid within the genome of an organism or cells thereof. For example, genetic modification can refer to alterations, additions, and/or deletion of genes. A genetically modified cell can also refer to a cell with an added, deleted and/or altered gene. A genetically modified cell from a genetically modified pig can be a cell isolated from such genetically modified pig. In some cases, a genetically modified cell of a pig comprises reduced expression of one or more genes as compared to a non-genetically modified counterpart animal. A non-genetically modified counterpart animal can be an animal substantially identical to the genetically modified animal but without genetic modification in the genome. For example, a non-genetically modified counterpart animal can be a wild-type animal of the same species as the genetically modified animal.

In another aspect, transgenic pigs suitable for use in xenotransplantation and methods of producing transgenic pigs suitable for use in xenotransplantation are provided. In particular, the present application describes the production of homozygous single and double transgenic mammals. As used herein, the term “transgenic” refers to a pig wherein a given gene has been altered, removed or disrupted.

As used herein, the term “knockout” refers to a transgenic non-human mammal wherein a given gene has been altered, removed or disrupted. The term is intended to encompass all progeny generations. Thus, the founder animal and all F1, F2, F3 and so on progeny thereof are included, regardless of whether progeny were generated by gene editing or somatic cell nuclear transfer (SCNT) from the founder animal or a progeny animal or by traditional reproductive methods. By “single knockout” is meant a transgenic mammal wherein one gene has been altered, removed or disrupted. By “double knockout” is meant a transgenic mammal wherein two genes have been altered, removed or disrupted. By “triple knockout” is meant a transgenic mammal wherein three genes have been altered, removed or disrupted. By “quadruple knockout” is meant a transgenic mammal wherein four genes have been altered, removed or disrupted. By “quintuple knockout” is meant a transgenic mammal wherein five genes have been altered, removed, or disrupted.

The transgenic mammal may have one or both copies of the gene sequence of interest disrupted. In the case where only one copy or allele of the nucleic acid sequence of interest is disrupted, the transgenic animal is termed a “heterozygous transgenic animal.” The term “null” mutation encompasses both instances in which the two copies of a nucleotide sequence of interest are disrupted differently but for which the disruptions overlap such that some genetic material has been removed from both alleles, and instances in which both alleles of the nucleotide sequence of interest share the same disruption. In various embodiments, disruptions of porcine B3gnt5 may occur in at least one cell of the transgenic animal, at least a plurality of the animal's cells, at least half the animal's cells, at least a majority of animal's cells, at least a supermajority of the animal's cells, at least 70%, 75%, 80%, 85%, 90%, 95%, 98%, or 99% of the animal's cells.

The species of the mammal of this invention is not restricted, as far as it is non-human. Farm animals and experimental animals are examples. More particularly, such animals as the pig, bovine, equine, ovine, goat, dog, rabbit, mouse, rat, guinea pig, and hamster are examples. Although the application describes a typical non-human animal (pigs), other animals can similarly be genetically modified. As mentioned above, the pig is desirable in organ transplantation to humans. As used herein, the term “pig” refers to any pig known to the art including, but not limited to, a wild pig, domestic pig, mini pigs, a Sus scrofa pig, a Sus scrofa domesticus pig, as well as in-bred pigs. Without limitation the pig can be selected from the group comprising Landrace, Yorkshire, Hampshire, Duroc, Chinese Meishan, Chester White, Berkshire Goettingen, Landrace/York/Chester White, Yucatan, Bama Xiang Zhu, Wuzhishan, Xi Shuang Banna, and Pietrain pigs. Porcine organs, tissues or cells include organs, tissues, devitalized animal tissues, and cells from a pig.

Transgenic pigs as described herein can be achieved by, for example, altering, removing, or otherwise disrupting B3gnt5 alleles, or replacement of B3gnt5 alleles with genetically modified sequences. In some cases, transgenic pigs are produced using homologous recombination and somatic cell nuclear transfer (SCNT) methods. Other methods for producing genetically modified non-human animals are generally known in the art, and are described in Sambrook et al, Molecular Cloning, A Laboratory Manual, Cold Spring Harbor Press, Cold Spring Harbor, N.Y. (1989), incorporated herein by reference. In some cases, genetic modifications are produced using a form of gene editing. The term “gene editing” and its grammatical equivalents as used herein refer to genetic engineering in which one or more nucleotides are inserted, replaced, or removed from a genome. For example, gene editing can be performed using a nuclease (e.g., a natural-existing nuclease or an artificially engineered nuclease). To date, several gene-knockout pigs have been generated using Zinc-finger nuclease (ZFN) and transcription activator-like effector nuclease (TALEN) gene-editing technologies.

In certain embodiments, Cas gene editing is performed using a CRISPR/Cas system (e.g., a type II CRISPR/Cas system). For example, a CRISPR/Cas system can be used to reduce expression of one or more genes in cells of a spheroid. In some cases, the protein expression of one or more endogenous genes is reduced using a CRISPR/Cas system. In other cases, a CRISPR/Cas system can be used to perform site specific insertion. For example, a nick on an insertion site in the genome can be made by CRISPR/Cas to facilitate the insertion of a transgene at the insertion site. Other methods of making genetic modifications suitable for use according to the methods provided herein include but are not limited to somatic cell nuclear transfer (SCNT) and introduction of a transgene. As used herein, the term “transgene” refers to a gene or genetic material that can be transferred into an organism or a cell thereof. Procedures for obtaining recombinant or genetically modified cells are generally known in the art, and are described in Sambrook et al, Molecular Cloning, A Laboratory Manual, Cold Spring Harbor Press, Cold Spring Harbor, N.Y. (1989), incorporated herein by reference.

In another aspect, provided herein are porcine organs, tissues, and cells obtained from genetically modified pigs described herein. In some cases, the porcine organs, tissues, and cells of a genetically modified pig are organs, tissues, and cells useful for transplantation including, without limitation, skin, a skin-related product, heart, liver, kidneys, lung, pancreas, thyroid, small bowel, and components thereof. As used herein, the term “skin related product” refers to products isolated from skin and products intended for use with skin. Skin related products isolated from skin or other tissues may be modified before use with skin. Skin related products include but are not limited to replacement dressings, burn coverings, dermal products, replacement dermis, dermal fibroblasts, collagen, chondroitin, connective tissue, keratinocytes, cell-free xenodermis, cell-free pig dermis, composite skin substitutes and epidermis and temporary wound coverings.

In another aspect, provided herein is transplant material encompasses organs, tissue and/or cells from an animal for use as xenografts. Transplant material for use, as xenografts, may be isolated from transgenic animals with decreased expression of B3gnt5, or from transgenic animals lacking B3gnt5. Transgenic transplant material from knockout pigs can be isolated from a prenatal, neonatal, immature or fully mature animal. The transplant material may be used as temporary or permanent organ replacement for a human subject in need of an organ transplant. Any porcine organ can be used including, but not limited to, the brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, small bowel, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes and lymph vessels.

To study the effects of various genetic modifications on human responses to porcine tissue transplants, it will be advantageous in some cases to generate transgenic pigs in which the genome has been genetically modified for reduced expression or to fully knock out expression of B3gnt5, as well as other xenoreactive antigens or immune-related molecules. For example, genetically modified porcine cells (e.g., LSECs, hepatocytes, fibroblasts) can be genetically modified using a CRISPR/Cas system to selectively reduce expression of one or more major histocompatibility complex (MHC) molecules (e.g., MHC I molecules and/or MHC II molecules) as compared to a non-genetically modified counterpart animal.

As used herein, the terms “synthetic” and “engineered” are used interchangeably and refer to a non-naturally occurring material that has been created or modified by a human (e.g., a genetically modified animal having one or predetermined engineered genetic modifications in its genome) or is derived using such material (e.g., a tissue or organ obtained from such genetically modified animal). In some cases, cells used to produce transgenic animals of this disclosure are porcine cells that contain one or more synthetic or genetically engineered nucleic acids (e.g., a nucleic acid containing at least one artificially created insertion, deletion, inversion, or substitution relative to the sequence found in its naturally occurring counterpart). Cells comprising one or more synthetic or engineered nucleic acids are considered to be engineered or genetically modified cells. As used herein, the term “engineered tissue” refers to aggregates of engineered/genetically modified cells.

Expression of a gene product is decreased when total expression of the gene product is decreased, a gene product of an altered size is produced, or when the gene product exhibits an altered functionality. Thus, if a gene expresses a wild-type amount of product but the product has an altered enzymatic activity, altered size, altered cellular localization pattern, altered receptor-ligand binding or other altered activity, expression of that gene product is considered decreased. Expression may be analyzed by any means known in the art including, but not limited to, RT-PCR, Western blots, Northern blots, microarray analysis, immunoprecipitation, radiological assays, polypeptide purification, spectrophotometric analysis, Coomassie staining of acrylamide gels, ELISAs, 2-D gel electrophoresis, in situ hybridization, chemiluminescence, silver staining, enzymatic assays, ponceau S staining, multiplex RT-PCR, immunohistochemical assays, radioimmunoassay, colorimetric analysis, immunoradiometric assays, positron emission tomography, fluorometric assays, fluorescence activated cell sorter staining of permeabilized cells, radioimmunosorbent assays, real-time PCR, hybridization assays, sandwich immunoassays, flow cytometry, SAGE, differential amplification, or electronic analysis.

Expression may be analyzed directly or indirectly. Indirect expression analysis may include but is not limited to, analyzing levels of a product catalyzed by an enzyme to evaluate expression of the enzyme.

As used herein, “as compared to” is intended encompass comparing something to a similar but different thing, such as comparing a data point obtained from an experiment with a knockout pig to a data point obtained from a similar experiment with a wild-type pig. The word “comparing” is intended to encompass examining the character, qualities, values, quantities or ratios in order to discover resemblances or differences between that which is being compared. Comparing may reveal a significant difference in that which is being compared. By “significant difference” is intended a statistically significant difference in results obtained for multiple groups, such as the results for a first aliquot and a second aliquot. Generally, statistically significance is assessed by a statistical significance test such as but not limited to the student's t-test, Chi-square, one-tailed t-test, two-tailed t-test, ANOVA, Dunett's post hoc test, Fisher's test and z-test. A significant difference between the two results may be results with a p<0.1, p<0.05, p<0.04, p<0.03, p<0.02, or p<0.01 or greater.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Nucleic acids and/or other moieties of the invention may be isolated. As used herein, “isolated” means separate from at least some of the components with which it is usually associated whether it is derived from a naturally occurring source or made synthetically, in whole or in part. Nucleic acids and/or other moieties of the invention may be purified. As used herein, “purified” means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

In another aspect, provided herein is a method of improving a rejection related symptom in a human subject. In some cases, the method comprises transplanting porcine transplant material having reduced levels of LNnT antigens into a human subject in need thereof, wherein a rejection related symptom is improved as compared to when porcine transplant material from a wild-type pig is transplanted into a human subject. Transplant rejection occurs when transplanted tissue, organs, cells or other biological material are not accepted by the recipient's body. In transplant rejection, the recipient's immune system attacks the transplanted material. Multiple types of transplant rejection exist and may occur separately or together. Rejection processes included but are not limited to hyperacute rejection (HAR), acute humoral xenograft rejection reaction (AHXR), thrombocytopenia, acute humoral rejection, hyperacute vascular rejection, antibody mediated rejection and graft versus host disease. “Hyperacute rejection” means rejection of the transplanted material or tissue occurring or beginning within the first 24 hours post-transplant involving one or more mechanisms of rejection. Rejection encompasses but is not limited to “hyperacute rejection,” “humoral rejection,” “acute humoral rejection,” “cellular rejection,” and “antibody mediated rejection.” Acute humoral xenograft reaction (AHXR) is characterized by a spectrum of pathologies including without limitation acute antibody mediated rejection occurring within days of transplant, the development of thrombotic microangiopathy (TMA), microvascular angiopathy, pre-formed non-Gal IgM and IgG binding, complement activation, microvascular thrombosis and consumptive thrombocytopenia within the first few weeks post-transplant.

In certain embodiments, xenotransplantation of a porcine organ, tissue, or cell from a transgenic pig described herein results in an improvement of one or more rejection related symptoms selected from a cellular rejection response related symptom, a humoral rejection response related symptom, a hyperacute rejection (HAR) related symptom, an acute humoral xenograft reaction rejection related symptom, and an acute vascular rejection response related symptom wherein one or more rejection related symptoms is improved as compared to when tissue from a wild-type swine is transplanted into a human. By “improving,” “bettering,” “ameliorating,” “enhancing,” and “helping” is intended advancing or making progress in what is desirable. It is also envisioned that improving a rejection related symptom may encompass a decrease, lessening, or diminishing of an undesirable symptom. It is further recognized that a rejection related symptom may be improved while another rejection related symptom is altered.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

The invention will now be illustrated by the following non-limiting Examples.

### Example 1

### Carbohydrate Antigen Microarray Analysis of Serum IgG and IgM Antibodies Before and After Adult Porcine Islet Xenotransplantation in Cynomolgus Macaques

Understanding the anti-carbohydrate antibody response toward epitopes expressed on porcine cells, tissues, and organs is critical to advancing xenotransplantation toward clinical application. In this study, we determined IgM and IgG antibody specificities and relative concentrations in five cynomolgus monkeys at baseline and at intervals following intraportal xenotransplantation of adult porcine islets. This study utilized a carbohydrate antigen microarray that comprised more than 400 glycoconjugates, including historically reported α-Gal and non-α-Gal carbohydrate antigens with various modifications. The elicited anti-carbohydrate antibody responses were predominantly IgM compared to IgG in 4 out of 5 monkeys. Patterns of elicited antibody responses greater than 1.5 difference (log 2 base units; 2.8-fold on a linear scale) from pre-serum to post-serum sampling specific for carbohydrate antigens were heterogeneous and recipient-specific. Increases in the elicited antibody response to α-Gal, Sd^{a}, GM2 antigens, or Lexis X antigen were found in individual monkeys. The novel carbohydrate structures Galβ1-4GlcNAcβ1-3Galβ1 and N-linked glycans with Manα1-6 (GlcNAcβ1-2Manα1-3)Manβ1-4GlcNAcβ structure were common targets of elicited IgM antibodies. These results provide important insights into the carbohydrate epitopes that elicit antibodies following pig-to-monkey islet xenotransplantation and reveal possible targets for gene editing.

### Introduction

Islet transplantation is a vital treatment option for patients in whom type 1 diabetes is complicated by recurrent severe hypoglycemia patients with severe glycemic instability. The effectiveness of transplantation of human islets in preventing severe hypoglycemia is unmatched; however, the shortage of human donors and complications of immunosuppression preclude its wider application in diabetes care. Genetically engineered pigs could provide an unlimited source of islets with low immunogenicity for xenotransplantation into patients with diabetes, thereby possibly offering a solution to the two main limitations of human islet transplantation.

Antibodies recognizing cell surface carbohydrates and proteins of xenogeneic grafts activate the classical complement pathway, contributing to hyperacute, acute vascular, and late rejection of the graft. The predominant carbohydrate xenoantigen is the galactose-α-1,3-galactose (Gala1-3Gal, α-Gal) epitope, which is synthesized by the α1,3-galactosyltransferase gene (GGTA1) and a target of hyperacute rejection (HAR). In addition to α-Gal, N-glycolylneuraminic acid (Neu5Gc) and the Sd^{a} glycan are also known xenoantigens that contribute to early antibody-mediated immune injury.

Recent progress in genome editing has paved the way for the creation of pigs whose cells have reduced levels of the known xenoreactive antigens, which facilitated prolonged xenograft survival. Nevertheless as-yet-discovered xenoantigens still prevented graft acceptance, and thus identifying additional novel xenoantigens will play an important role in developing strategies for establishing long-term graft function. A detailed analysis of natural IgM and IgG antibody specificity and concentration from naïve cynomolgus macaques was performed utilizing a novel carbohydrate microarray (glycan microarray), in which more than 400 glycoconjugates and carbohydrates are attached to solid supports. Use of high-resolution carbohydrate microarray has the potential to elucidate the specific carbohydrate target of islet xenograft rejection by directly comparing naïve and posttransplant sera from individual monkeys receiving immunosuppression. The current study revealed that (i) the elicited anti-carbohydrate antibody responses were predominantly IgM compared to IgG; (ii) patterns of antibody responses were heterogeneous and individually specific; (iii) the novel carbohydrate structures Galb1-4GlcNAcb1-3Galb1 and Mana1-6(GlcNAcb1-2Mana1-3)Manb1-4GlcNAcb were common targets of elicited IgM. This study reveals targets of genetic engineering that give rise to donor animals that have lower antibody binding, and reveals novel targets of screening tools to select low antibody reactivity recipients.

### Methods

### Animals

A detailed analysis for natural IgM and IgG antibody levels of naïve cynomolgus macaques (Macaca fascicularis) of Mauritian origin and the background and housing conditions of the monkeys was done previously. Five male monkeys with an median age of 5.3 years (range: 4.4-5.8) and median weight of 5.0 kg (4.4-7.6) with blood type O (n=2), B (n=2), and AB (n=1) were recipients of islet xenotransplantation. One monkey (13GP08, O) received islets from GGTA1-KO pigs, and the others received islets from wild-type pigs. All animal procedures were approved by the University of Minnesota Institutional Animal Care and Use Committee and conducted in compliance with the Animal Welfare Act and adhere to principles stated in the Guide for Care and Use of Laboratory Animals.

### Pig Islet Isolation and Transplantation

Pig islets were isolated. Briefly, donor pancreases were retrieved from exsanguinated pigs, dissected, distended intraductally with collagenase and neutral protease, and dissociated using the automated method at 28° to 32° C. Liberated islets were separated from non-islet tissue on continuous density gradients on a Cobe 2991 cell separator and cultured free-floating in Medium 199 for 7 days before being infused intraportally through an indwelling catheter at a dose of 25,000 islet equivalents per kg into streptozotocin (100 mg/kg i.v.)-diabetic monkeys. Immunosuppression was administered to 13GP08, 12JP01, and 13CP10 recipients with the addition of α-CD20 during induction. Recipients 13CP03 and 13GP10 received α-CD25, CTLA4-Ig, sTNFR (etanercept, Enbrel®), and αIL-6R (tocilizumab, Actemra®) with the addition of maintenance antagonistic anti-CD40 mAb 2C10R4, provided by the NIH Nonhuman Primate Reagent Resource and Rapamycin (Rapamune®). Recipient monkeys received regular clinical and laboratory assessments. Posttransplant serum samples were collected at the time of clinical rejection and confirmed by histopathologic analysis.

### Carbohydrate Microarray Binding Assay

All sample analyses were performed at the Chemical Biology Laboratory, Center for Cancer Research, National Cancer Institute (Frederick, MD). Carbohydrate microarray fabrication was performed, and variability of the microarray assay was evaluated. Briefly, serum samples were profiled on a carbohydrate array (version A411) containing 408 carbohydrates and glycopeptides that were conjugated to albumin to produce neoglycoproteins. The number after the abbreviation indicates the average number of carbohydrate per molecule of albumin. Each monkey serum sample was assayed in two separate wells for IgG and two separate wells for IgM. All clinical/demographic information was blinded during the profiling of serum samples.

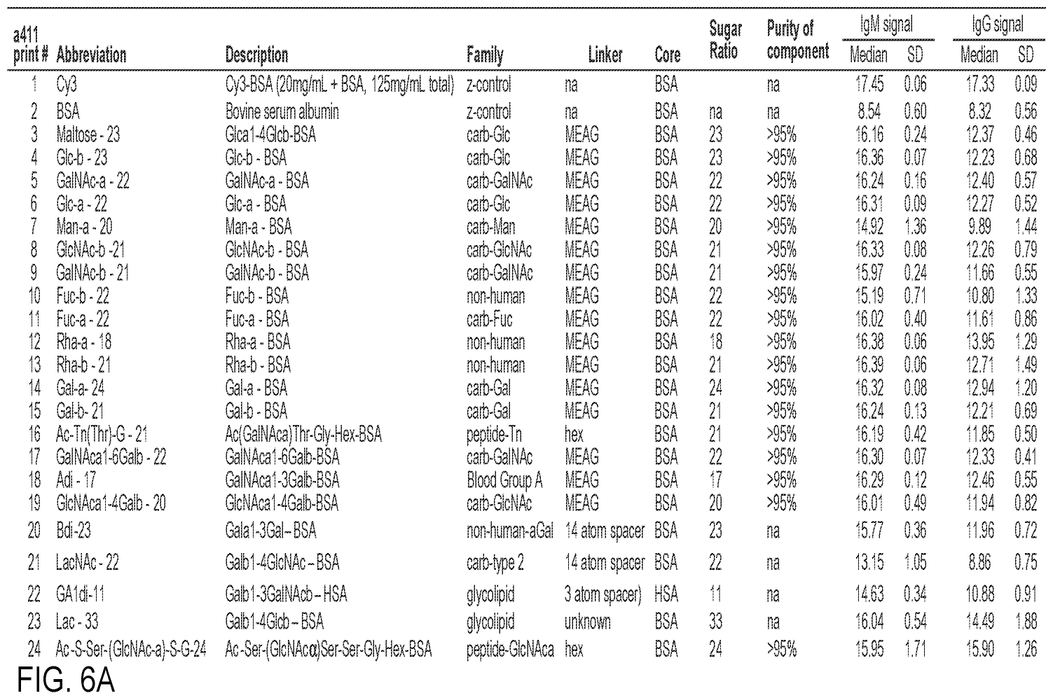

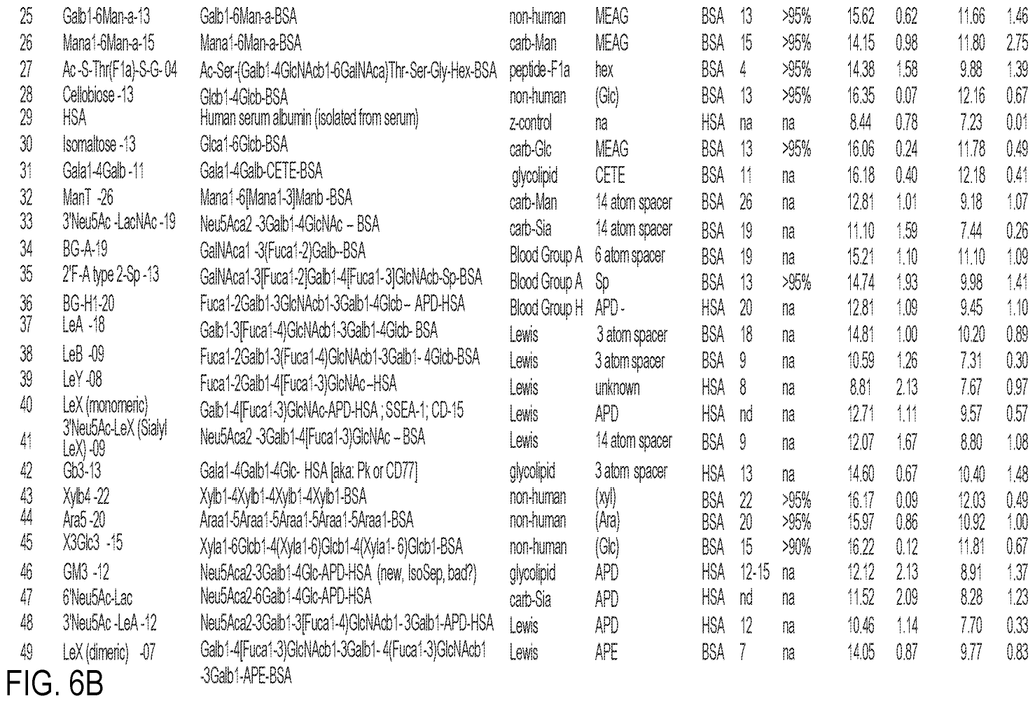

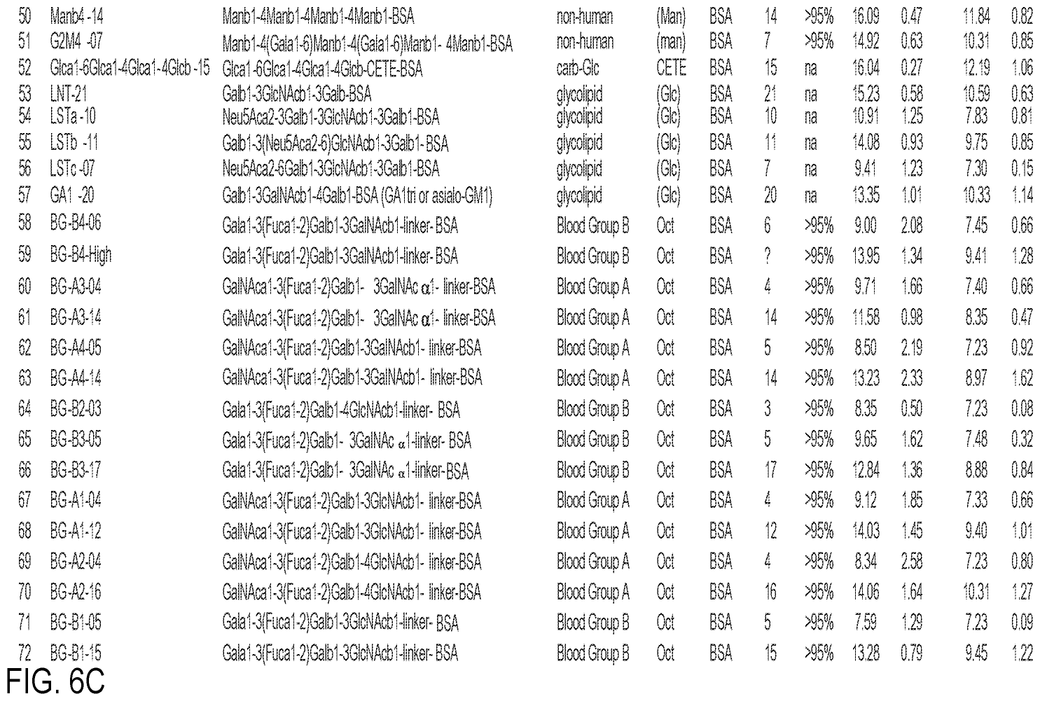

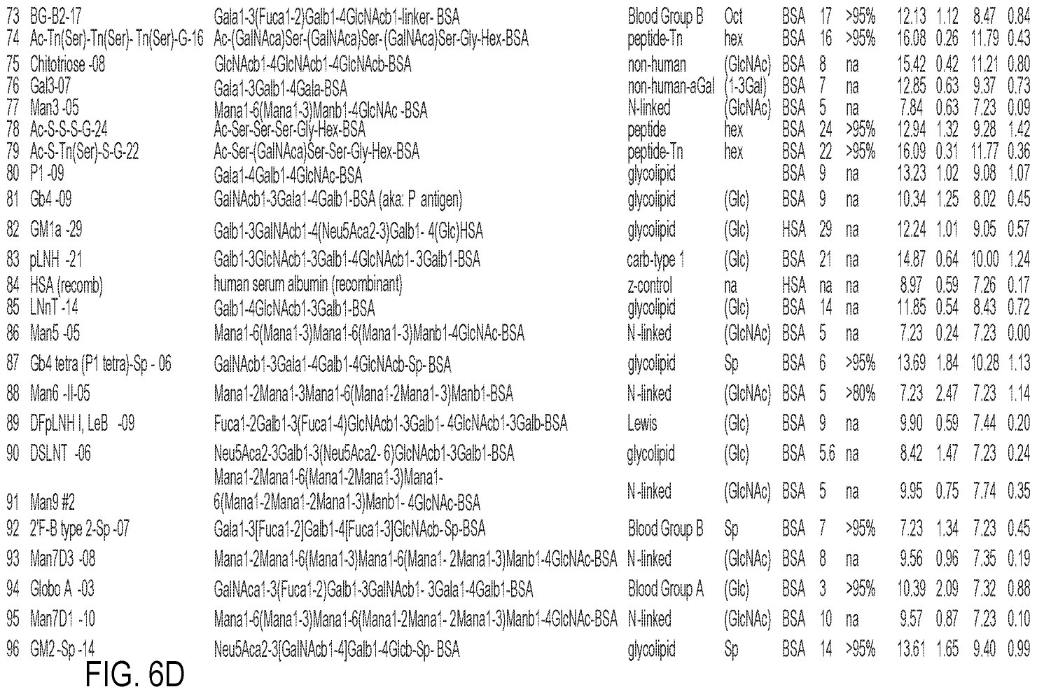

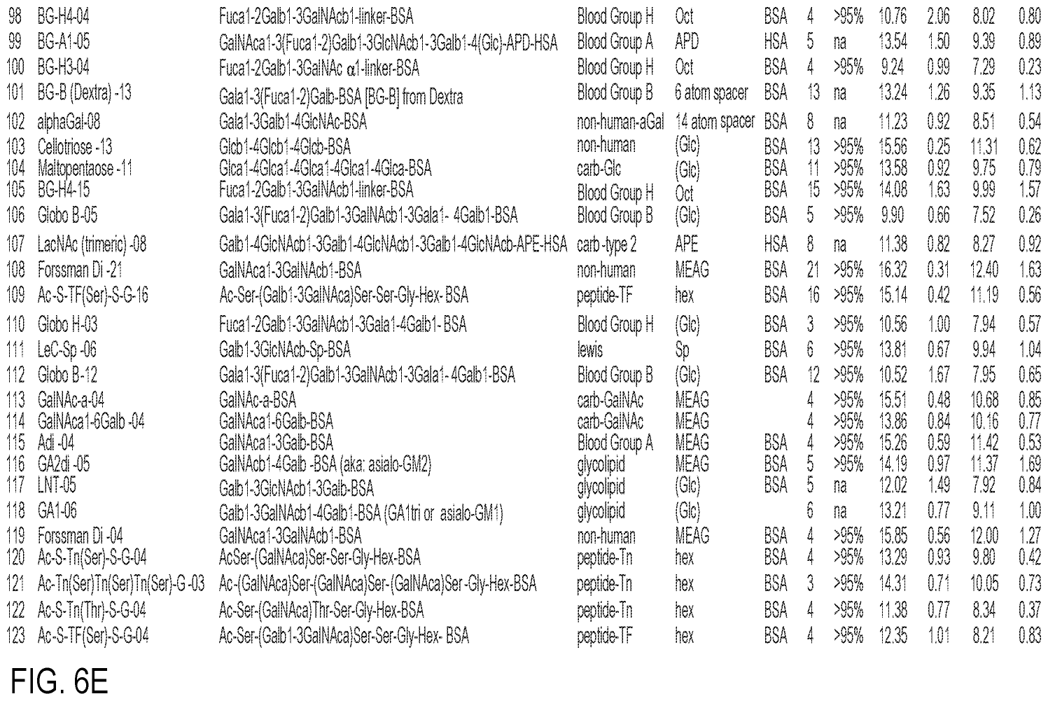

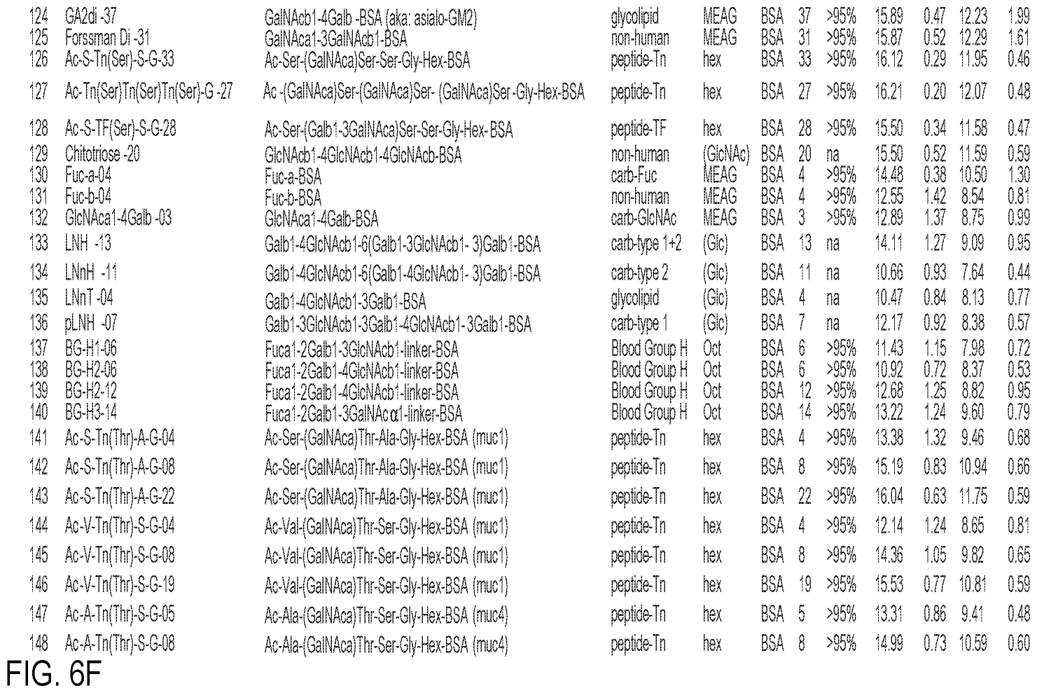

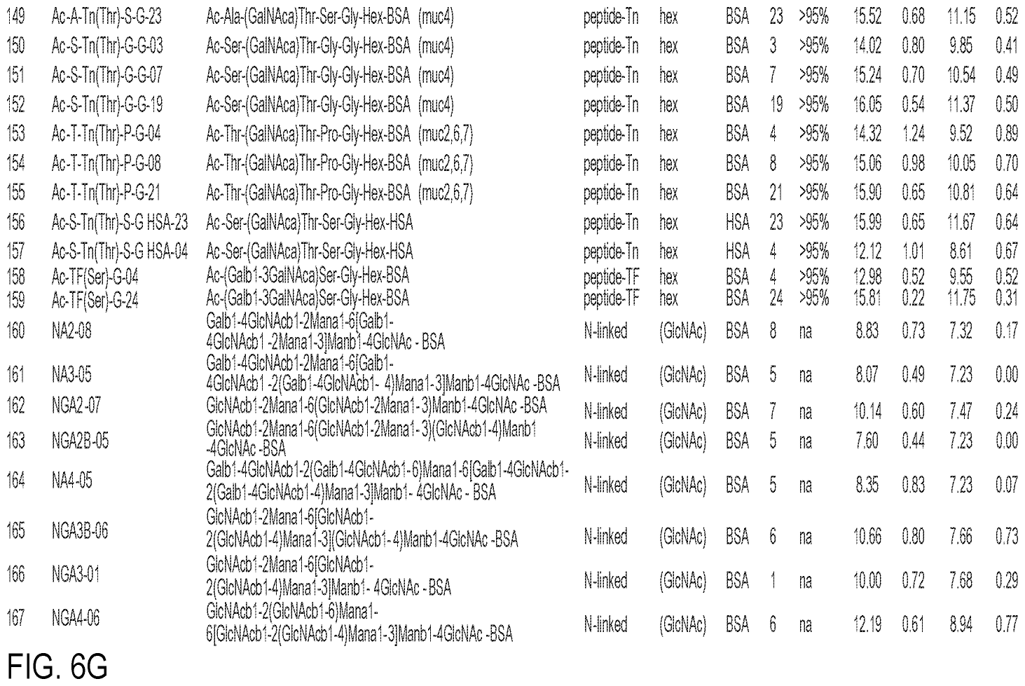

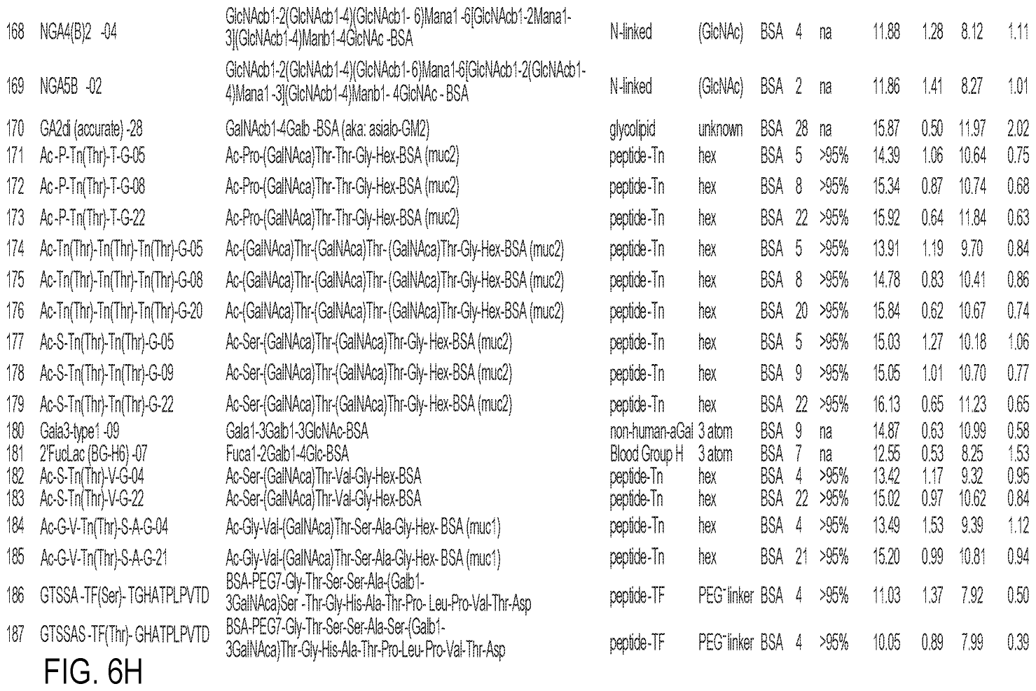

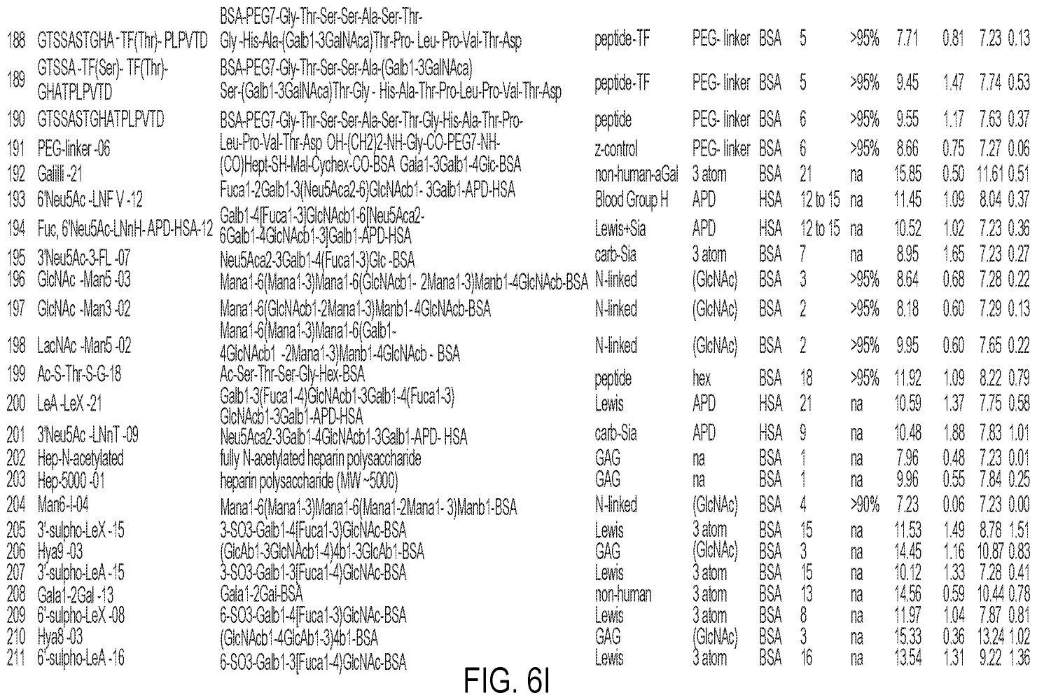

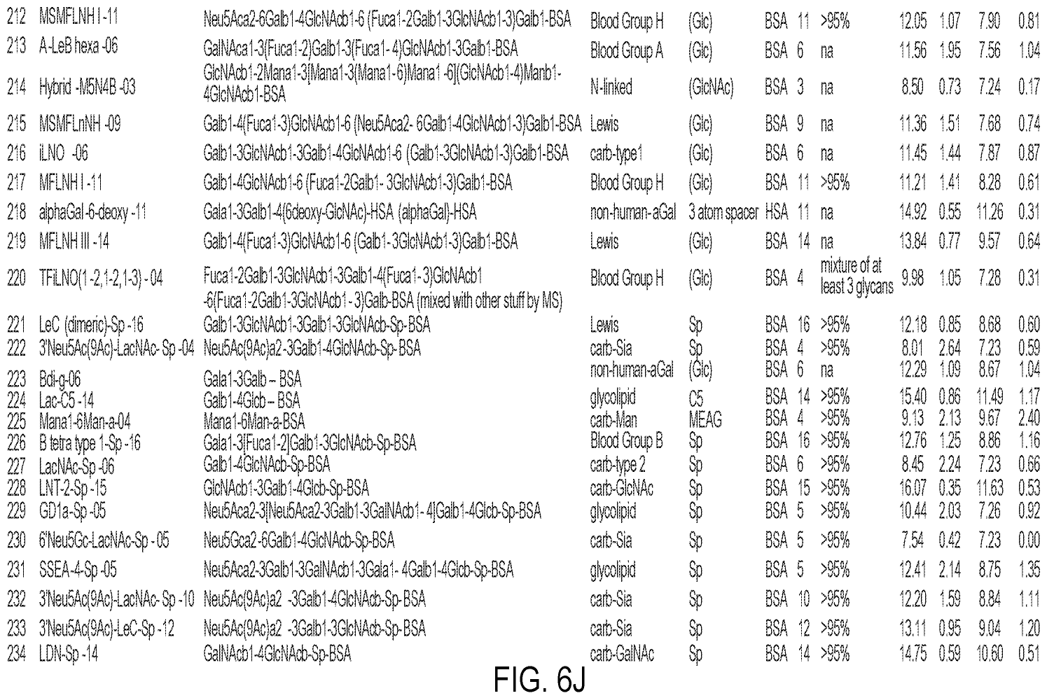

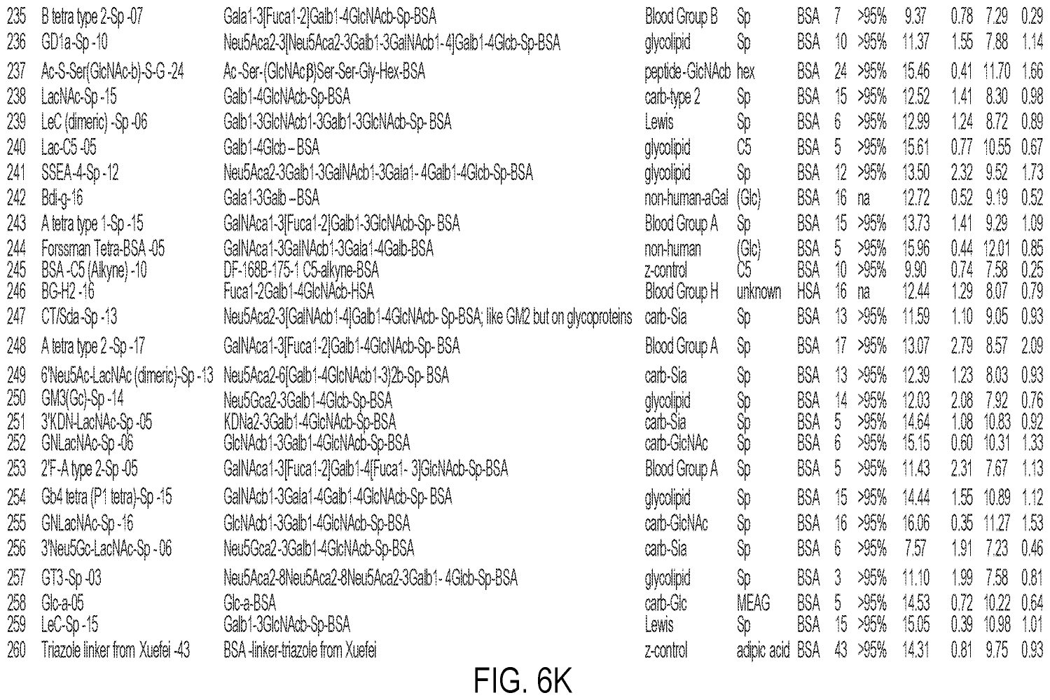

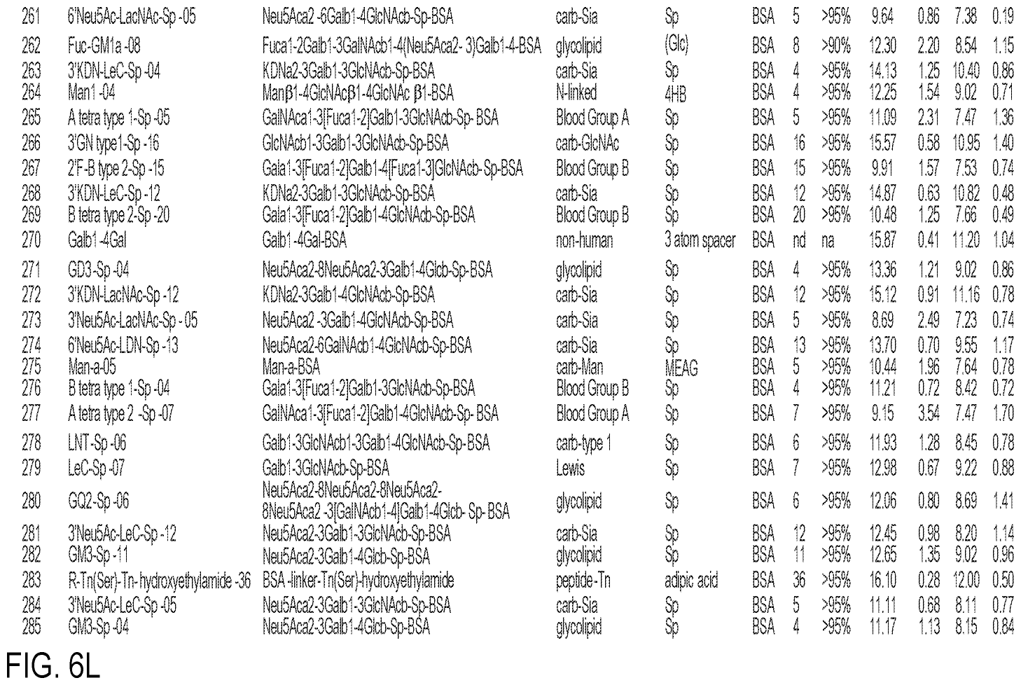

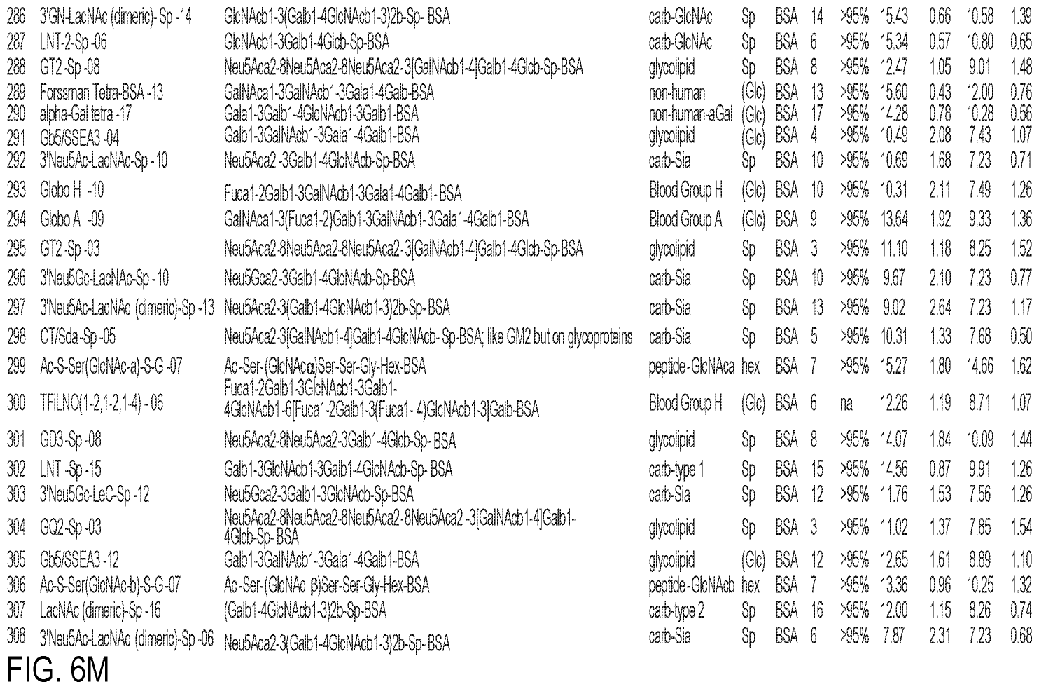

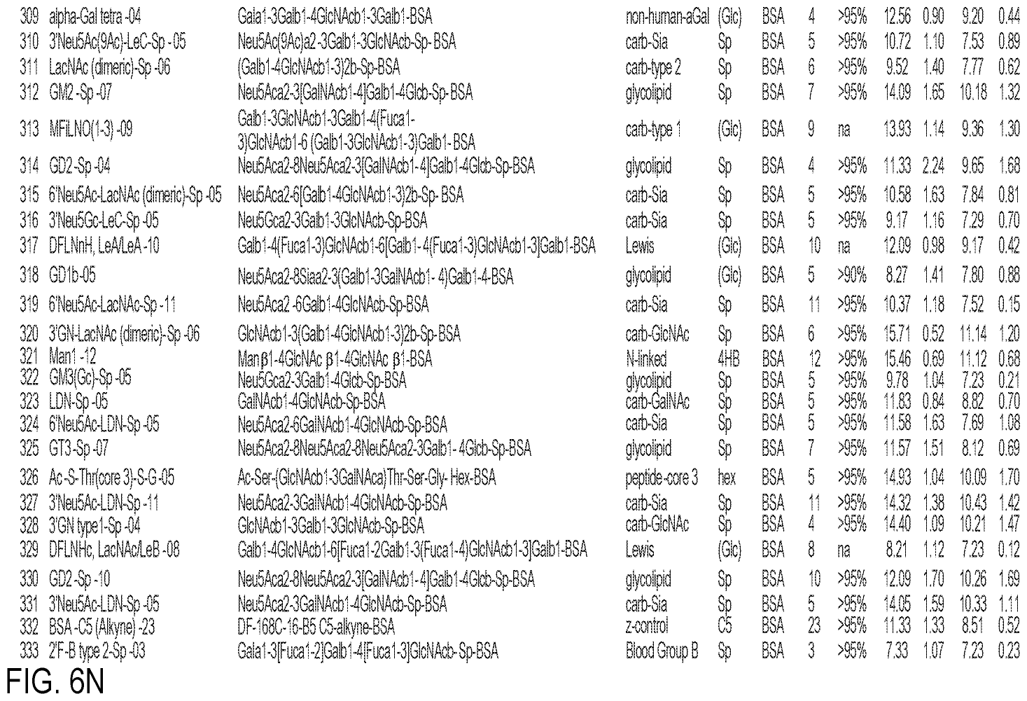

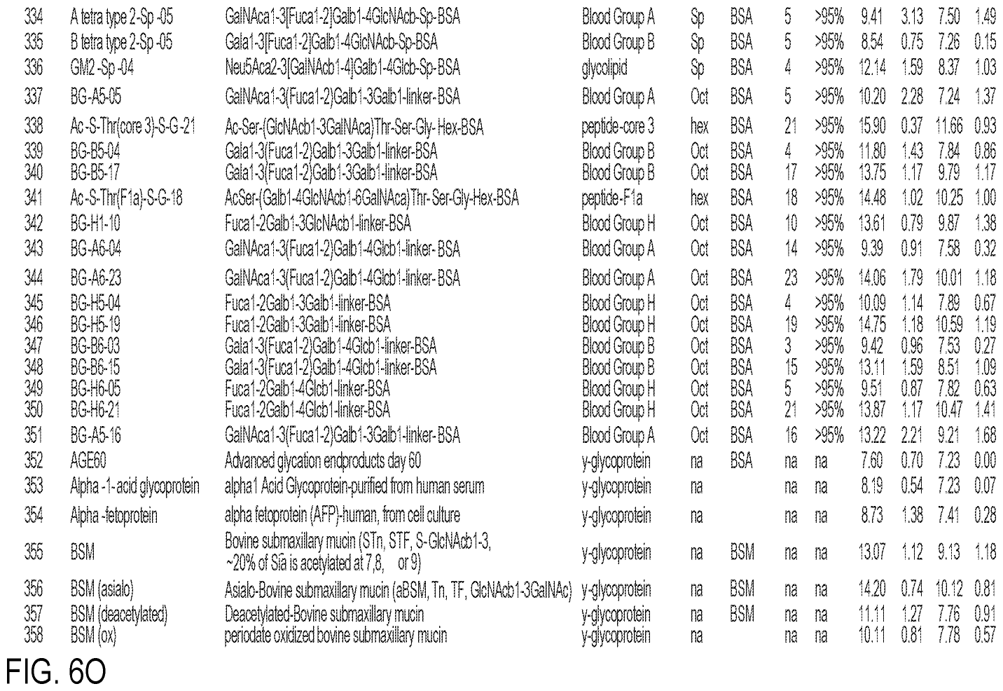

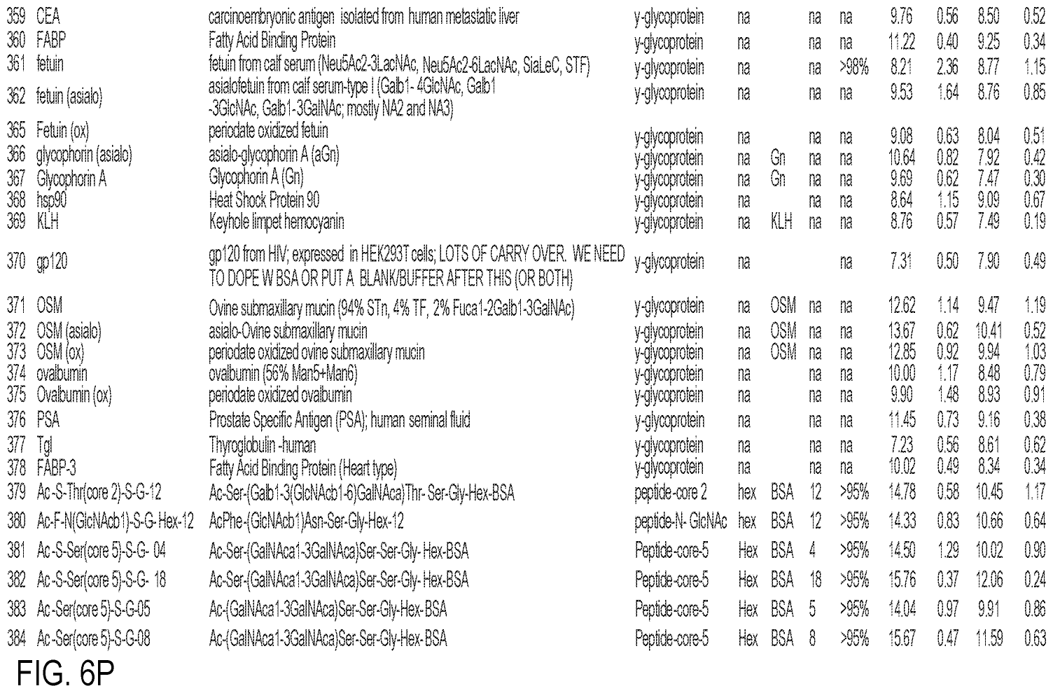

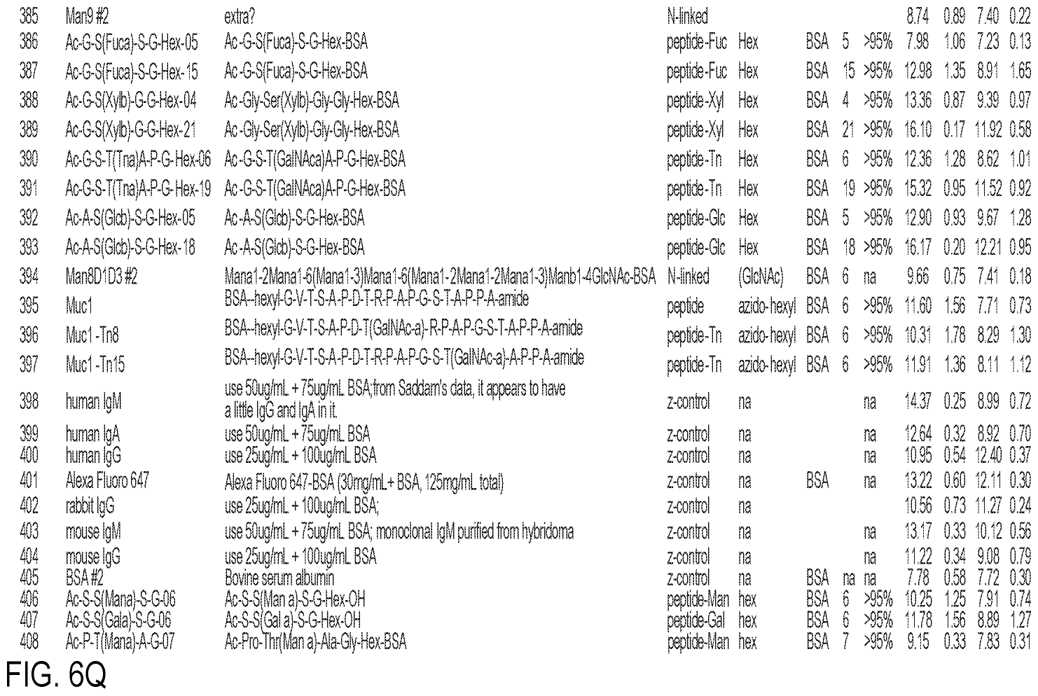

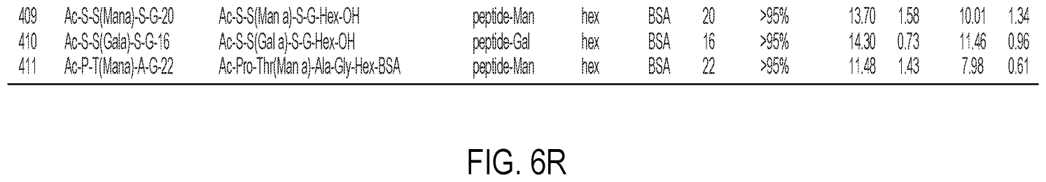

Slides were scanned at 5 m resolution with an InnoScan 1100 AL fluorescence scanner (Innopsys, Carbonne, France), and image analysis was carried out with Genepix Pro 6.0 analysis software (Molecular Devices Corporation, Union City, CA). To minimize the impact of noise on our comparisons, spots with intensity lower than 150 (½ the typical background signal when analyzing IgM and IgG at 1:50) were rounded to 150. The average of duplicate spots was calculated to obtain a normalized value to the reference samples and shown in a log-transformed (base 2) form, which enables a direct comparison of values from one experiment to another. Full microarray data can be found in FIGS. 6A-6R.

### Results

### Anti-Carbohydrate Antibody Signals in Posttransplant Sera

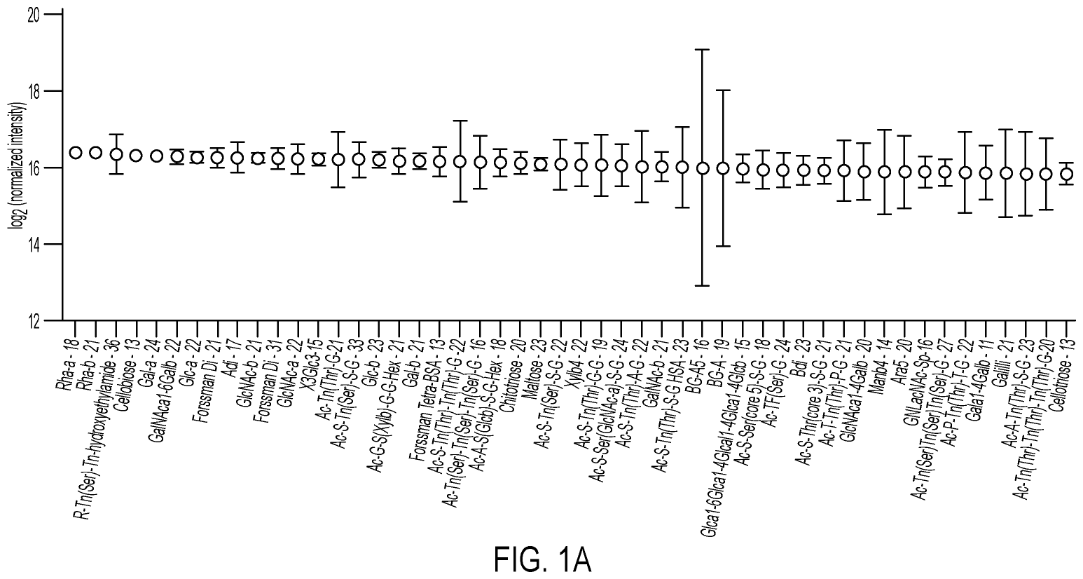

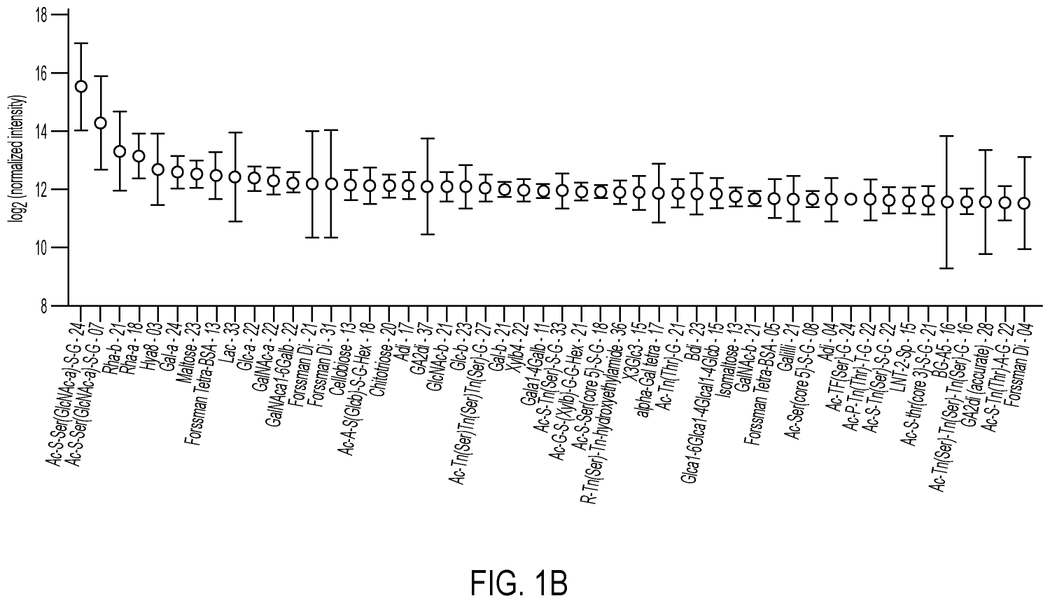

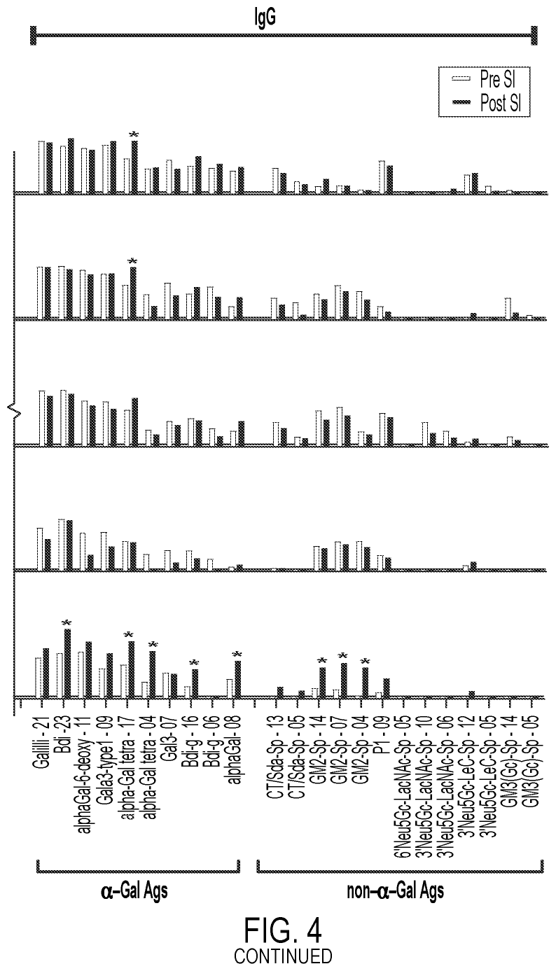

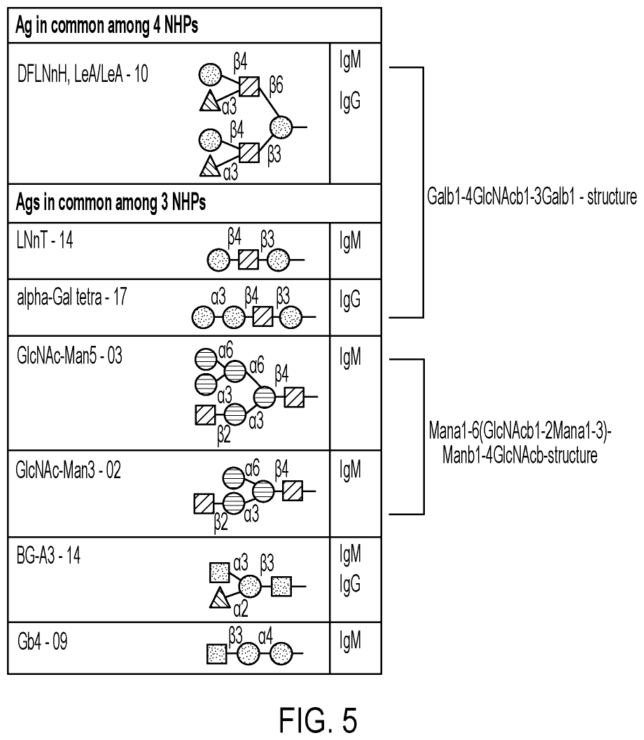

The median of IgM and IgG signals were 12.50 (7.23-16.40) and 9.23 (7.23-15.53), respectively. The distribution of 50 highest antibody signals in the posttransplant sera for each array component (n=408) is shown in FIG. 1A (IgM) and FIG. 1B (IgG). The details of signal intensities are shown in FIG. 5. The distribution of anti-carbohydrate antibody signal intensity in the sera was mostly similar to that of naïve sera in the previous report, but blood group A antigens (BG-A5-16, and BG-A-19) were listed in the posttransplant IgM and IgG repertoires with a wide distribution (FIGS. 1A-1B).

### Comparison of Antibody Signals Between Naïve and Posttransplant Sera

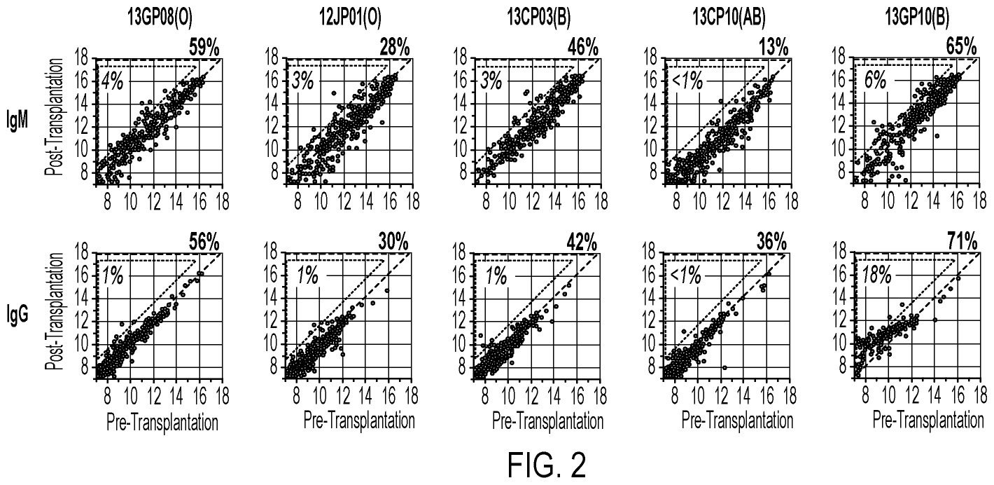

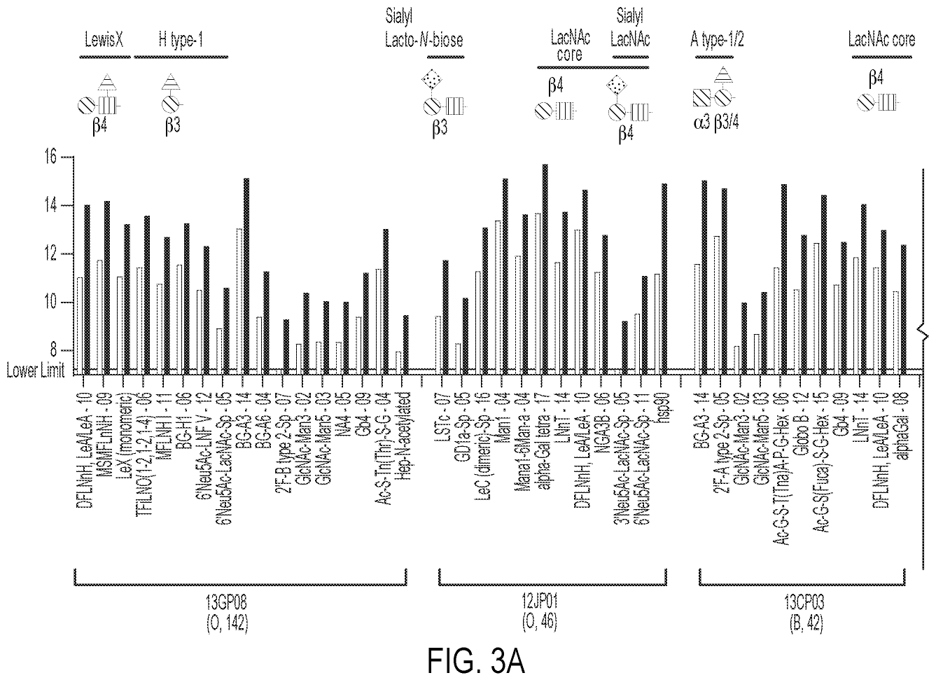

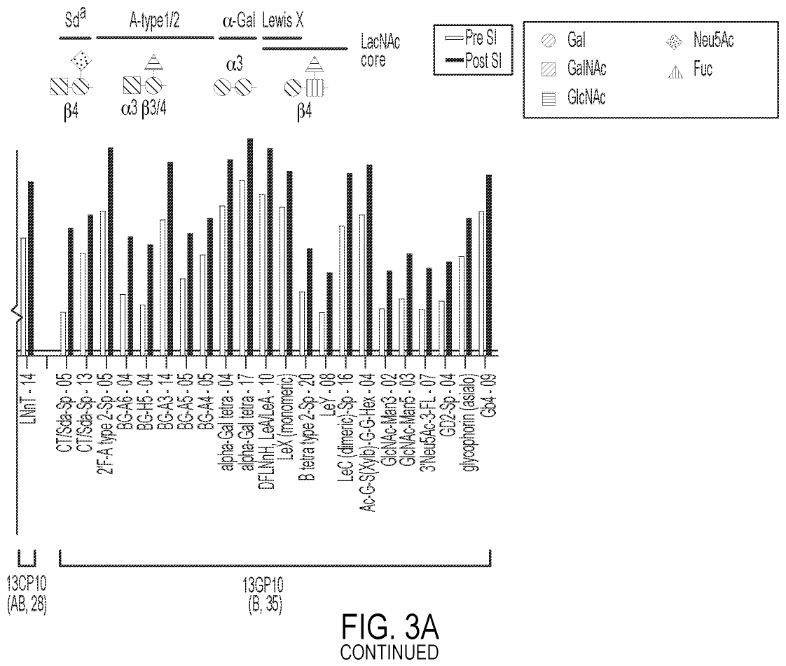

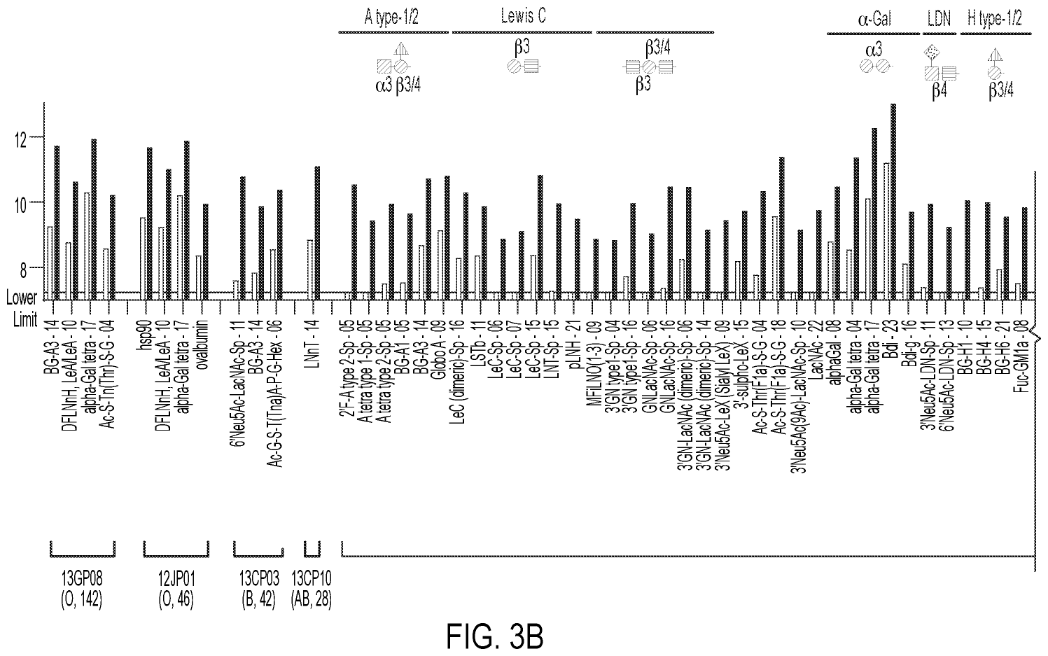

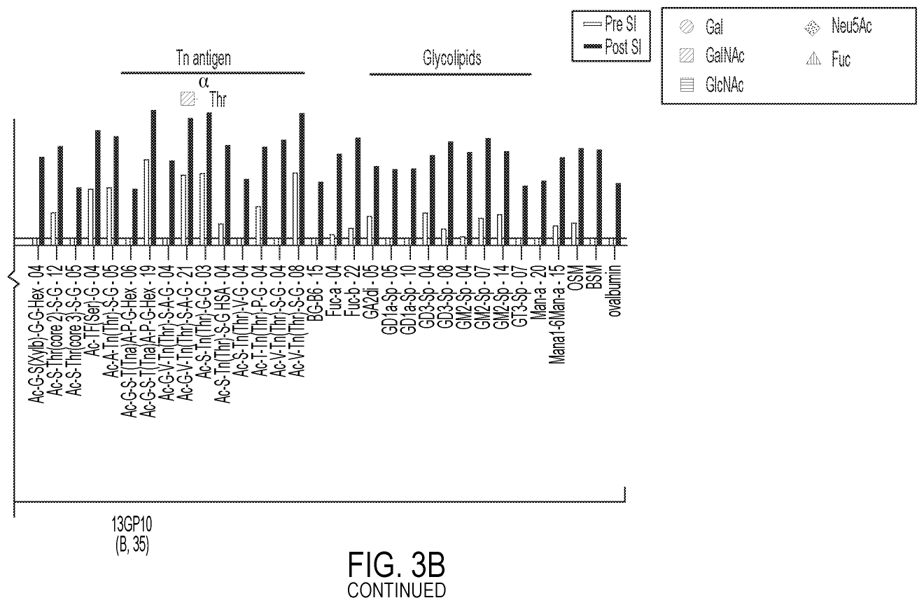

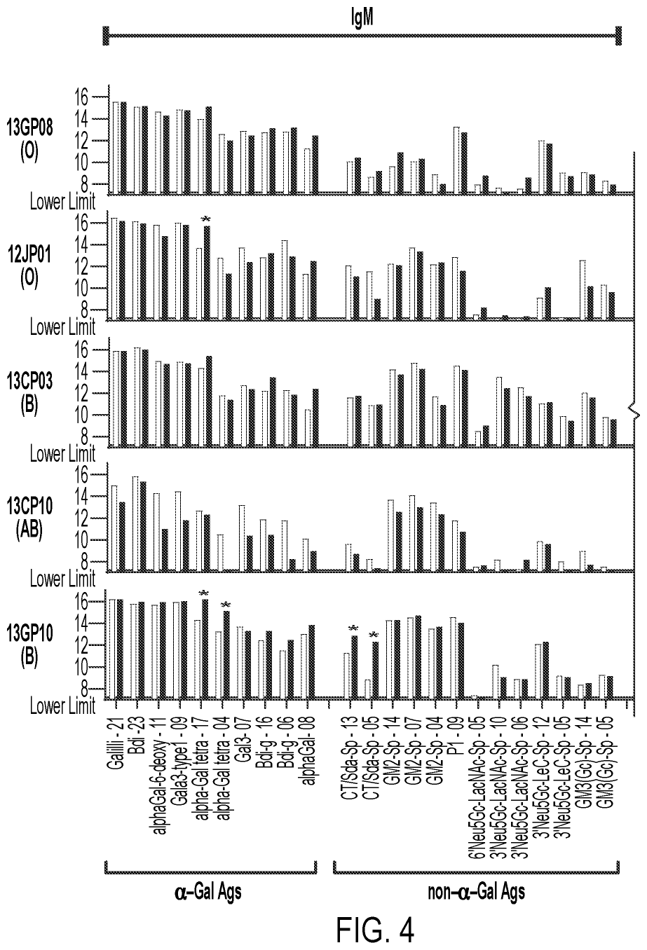

The comparison of IgM and IgG signal intensities between pretransplant naïve sera and posttransplant sera are shown in FIG. 2. The changes of normalized signal intensity ≥1.5 (log base 2 units; 2.8-fold on a linear scale) from pretransplant naïve serum IgM and IgG were considered significant. The threshold was determined by the previous analysis showing that the incidence of 1.5 signal intensity difference between the two time points in one-tailed test were approximately 1% for both IgG and IgM antibodies. The carbohydrates with a significant difference were grouped by epitope types and listed in FIGS. 3A-3B. The analysis of IgM and IgG signal intensities for previously reported α-Gal and non-α-Gal carbohydrate antigens were summarized in FIG. 4.

Monkey 13GP08 (Blood group O). The serum sample was obtained at sacrifice on day 142 after loss of function of GGTA1-KO porcine islets. Anti-carbohydrate antibodies exhibited a signal intensity rise against approximately 60% of all carbohydrates in both IgG and IgM (FIG. 2). Antibody against 17 carbohydrates were significantly elevated (cutoff of 1.5), which included carbohydrates with Lewis X and blood group H type-1 structures in IgM, whereas a specific trend was not observed in the IgG repertoire (FIGS. 3A-3B). One antibody toward an α-Gal epitope (alpha-Gal tetra-17) was elevated in IgG, but antibody toward other related α-Gal epitopes was not elevated in IgM and IgG repertoires (FIG. 4).

Monkey 12JP01 (Blood group O). The most unique carbohydrate reactivity detected in the sera collected at sacrifice on day 46 after rejection of functioning islets was a significant response against Sialyl Lacto-N-biose and Sialyl N-acetyllactosamine (LacNAc) structures (Neu5Aca2-6Galb1-3/4GlcNAc) in IgM (FIGS. 3A-3B). This monkey exhibited an increase in signal intensity towards approximately 30% of carbohydrates in both IgM and IgG repertoires (FIG. 2). Elicited antibody from 12JP01, as in 13GP08, recognized the alpha-Gal tetra-17 carbohydrate in addition to previously unrecognized non-α-Gal carbohydrate structures.

Monkey 13CP03 (Blood group B). The post-transplantation serum sample was taken on day 42 after primary non-function. Antibody from 13CP03 revealed a vigorous anti-blood group A antibody response and anti-LacNAc core structure response (FIGS. 3A-3B). The antibody signal intensities against blood group A antigens was significant (15.04-15.86) in the IgM repertoire. No specific trend was not observed in the IgG repertoire.

Monkey 13CP10 (Blood group AB). The serum sample was taken on day 28 at sacrifice after primary nonfunction. 13CP10 received islets from the same donor as 12JP01. In contrast to the other monkeys, a majority of carbohydrate signal intensities were decreased in both IgM and IgG repertoires (FIG. 2). However, a significant signal increase against LNnT-14 (Lacto-N-neotetraose, Galb1-4GlcNAcb1-3Galb1-BSA) was detected in both IgM and IgG repertoires (FIGS. 3A-3B). The LNnT carbohydrate was also elicited in 12JP01 and 13CP03. However, LNnT-04, which possesses the same epitope but in lower density, was not recognized by antibody in monkeys suggesting that antigen density is important in recognition.

Monkey 13GP10 (Blood group B). The post-transplantation serum sample was obtained on day 35 after primary nonfunction. The IgM binding significantly increased 65% and IgG increased 71% (FIG. 2). The elicited antibodies included Sd^{a} antigen, blood group A antigen, α-Gal antigen, and Lewis X antigen. The GM2 antigen that shares the Sd^{a} epitope [Neu5Aca2-3(GalNAcb1-4)Gal] showed a signal increase in the IgG repertoire. The GlcNAcb1-3Galb1-3/4GlcNAc structure, LDN, and Tn antigen carbohydrate structures also showed increases in the IgG response.

### Carbohydrate Antigens Elicited in Common Among Sera of Five Monkeys

Table 1 summarizes the carbohydrate targets of anti-carbohydrate antibodies elicited among five monkeys. The LNnT epitope (Galb1-4GlcNAcb1-3Galb1), shared among DFLNnH LeA/LeA, LNnT, and alpha-Gal tetra epitopes, elicited antibody in all monkeys. Two N-linked glycans in the table shared the GlcNAc-Man3 structure [Mana1-6(GlcNAcb1-2Mana1-3)Manb1-4GlcNAcb]. The signal intensities against the LNnT epitope were stronger than those against the GlcNAc-Man3 structures (FIGS. 3A-3B). No elicited anti-Neu5Gc response was detected in monkeys (FIG. 4).

### Discussion

Carbohydrate antigens on the surface of pig cells are major antigens in xenotransplantation. Genetic deletion of the enzymes that mediate the creation of those structures has reduced antibody binding and thereby prevented HAR. Yet flow cytometric cross match analysis suggests that additional antibody targets continue to contribute to xenograft rejection. The current carbohydrate antigen microarray study lead to the conclusion that the antibody responses against carbohydrate epitopes after intraportal xenotransplantation of adult pig islets into immunosuppressed cynomolgus monkeys: (i) are predominantly IgM compared to IgG in 4 out of 5 monkeys; (ii) are unique and specific to individual monkeys; only 1 out of 5 monkeys (13GP10) transplanted wild-type adult porcine islet had a statistically significant response against α-Gal and Sd^{a} antigens; (iii) yet the novel carbohydrate antigen structures LNnT (Galb1-4GlcNAcb1-3Galb1) and GlcNAc-Man3 [Mana1-6(GlcNAcb1-2Mana1-3)Manb1-4GlcNAcb] were targets of elicited IgM antibodies in 5 out of 5 and 3 out of 5 monkeys, respectively.

A detailed characterization of the elicited human antibodies after transplantation of porcine fetal islet-like cell clusters using a carbohydrate array system was previously performed. The present array study assessed sera after adult porcine islet transplantation and revealed novel antibody responses including those against LNnT and GlcNAc-Man3 structures, which were not found earlier. The present study suggested that the elicited antibody response to LNnT is density specific; only directed toward LNnT with higher density (LNnT-14 and DFLNnH, LeA/LeA-10) but not with lower density (LNnT-04). As antibodies achieve tight binding through formation of multivalent complexes, the density and the spacing between glycans affect the ability to form a multivalent interactions. It has been suggested that genetic modification of GGTA1 and CMAH could interfere with normal carbohydrate synthesis and cause accumulation of incomplete carbohydrate components including LNnT and high mannose incomplete N-glycans, presumably with a greater response to these structures in genetically modified donors. The presence of anti-LNnT (Galb1-4GlcNAcb1-3Galb1) and anti-GlcNAc-Man3 [Mana1-6(GlcNAcb1-2Mana1-3)Manb1-4GlcNAcb] antibodies was previously measured in human sera using the same glycan array used in the present study. Humans naturally have antibodies against the LNnT structure called “cold agglutinins” and the antibody has been thought to be irrelevant at body temperature and does not influence the outcome of the xenotransplantation. The successful generation of a LNnT deficient mouse was reported by deletion of β1,3-N-acetylglucosaminyltransferase 5 gene (B3gnt5). Deletion of this gene may be helpful in eliminating the antigenicity of the xenograft.

Although about 80% of human anti-porcine natural antibody is directed toward the α-Gal antigen that directly contributes to HAR, previous reports have shown that adult islet cells express negligible amount of α-Gal antigen, and a long-term reversal of diabetes is achieved after islet xenotransplantation in wild type pig-to-NHP models. However, in the current study, 3 out of 5 monkeys, including the recipient of islets from GGTA1-KO pigs, had elicited antibody responses against the same α-Gal antigen (alpha-Gal tetra, Gala1-3Galb1-4GlcNAcb1-3Galb1). This is likely a part of the antibody responses against Galb1-4GlcNAcb1-3Galb1 core, since antibody response against other α-Gal epitopes were not observed (FIG. 4). Sd^{a} antigen, identified as non-α-Gal antigen and validated by deletion of the B4GALNT2 gene, revealed that the elimination of the Sd^{a} epitope significantly reduced the level of human antibody to pig cells. The present work succeeded in showing basal levels of specific antibody binding to Sd^{a} and related GM2 antigens. A significant increase in the elicited antibody response, however, was observed in only one monkey (13GP10). Elicited antibody responses were also observed against blood group A antigens though not against B antigens (FIGS. 1A-1B and FIGS. 3A-3B). It was previously reported that there are only A or O blood types in pigs. Although blood-type testing of donor pigs was not currently done at the time these transplants occurred, it was assumed that the sensitization could occur from donor pig antigens. Cynomolgus macaques express the Neu5Gc epitope, which is analogous to pig but naturally absent in humans. As expected, epitopes with terminal Neu5Gc did not show a significant antibody response (FIG. 4). Statistically significant elicited antibody responses were predominantly IgM except 13GP10 (FIGS. 3A-3B). Increases in IgG as compared to IgM for the same glycan structure were observed, such as LNnT (e.g. 13CP10), suggesting that there is evidence of class switching yet further analysis is required to draw significant conclusions.

Recently, alternative and novel immunological pathways have been discovered and utilized to improve islet transplantation outcomes. Multidisciplinary approaches using these new immunoregulatory technologies in combination with optimized recipients with low reactivity towards xenoantigens will make it possible to progress clinical islet xenotransplantation to reality.

In conclusion, the present novel glycan array analysis of anti-porcine antibodies highlights the important implications for understanding the specificity of pre-existing and elicited antibody responses that may contribute to rejection. A heterogeneous and individually specific elicited antibody response suggests that the careful selection of recipients for low reactive antibody could positively affect outcomes in xenograft studies.

### Example 2

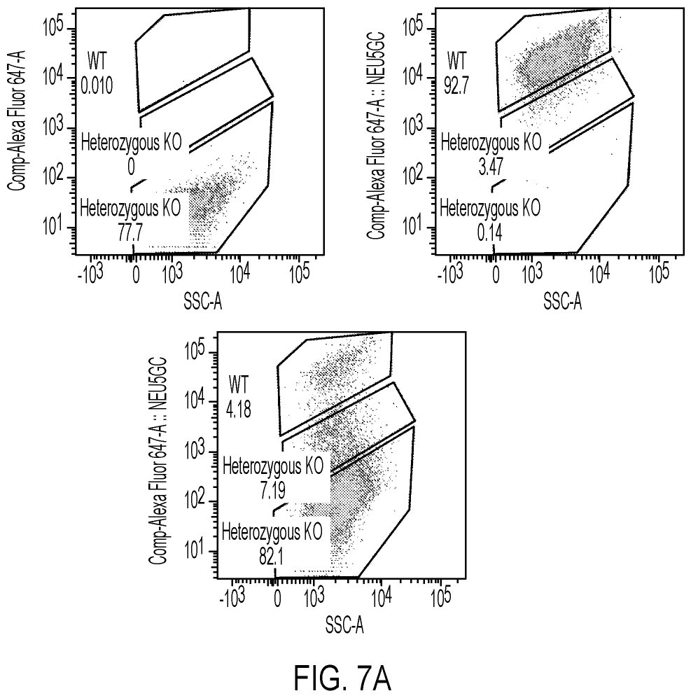

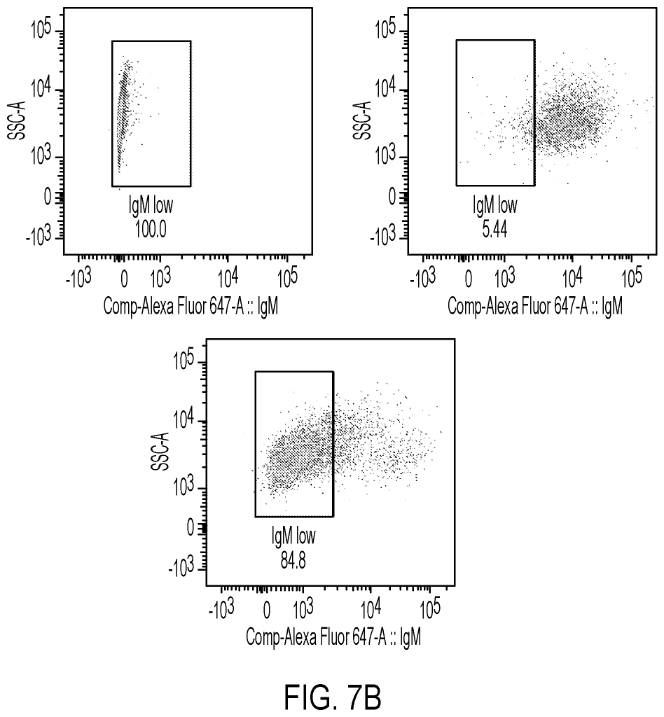

Fibroblasts cell transfected with Cas12 and guide RNA for GGTA1, CMAH, B4GalNT2, and B3GNT5 (FIGS. 7A-7B). Cell sorting for Neu5GC negative cells was performed at 7 days after transfection (FIG. 7A). Genetics in gates are confirmed by sanger sequencing. The B3GNT5 #6 guide RNA is ttctgcagttcaaatgggcaa. Cell sorting for monkey serum IgM low cells was performed at 14 days after transfection (FIG. 7B).

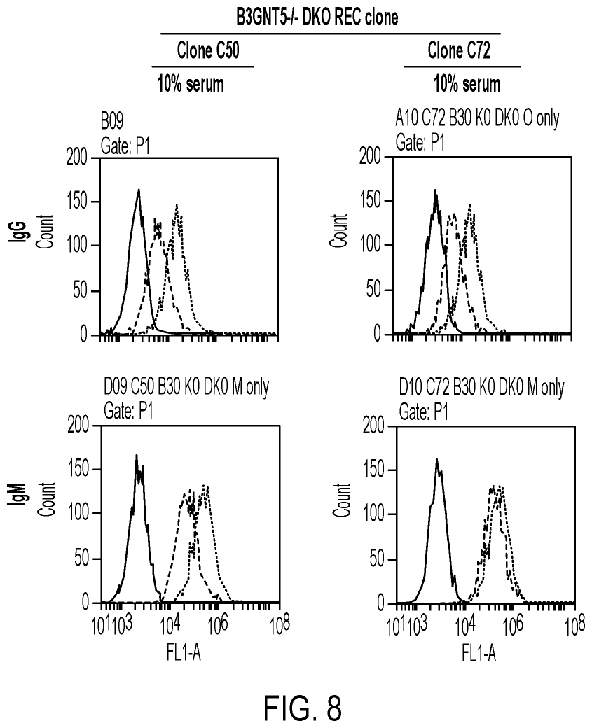

Crossmatch for B3GNT5^{-/-} deficient cells using Rhesus serum was performed. Incubation of B3GNT5^{-/-} sorted engineered cells with 10% serum showed less binding of antibody known to cause antibody mediated rejection (FIG. 8).

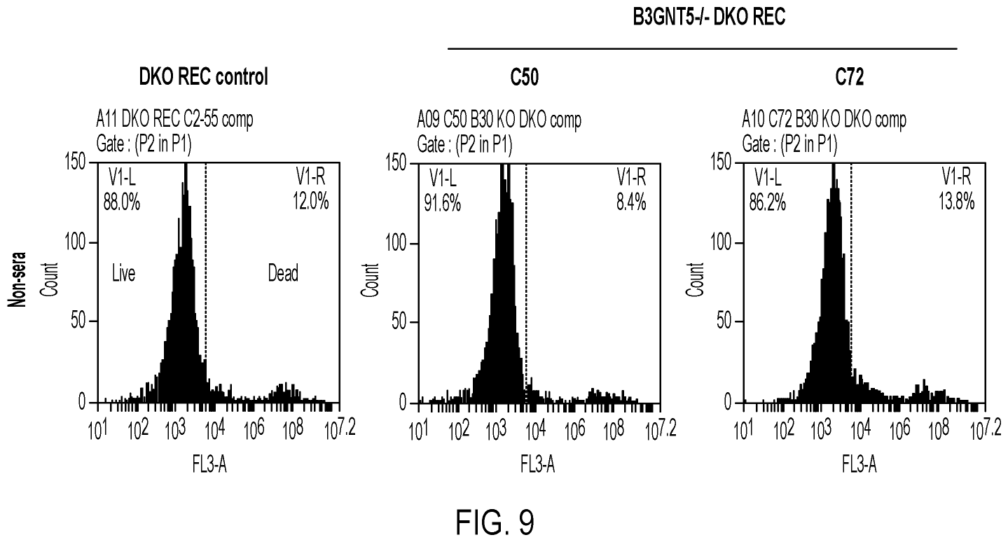

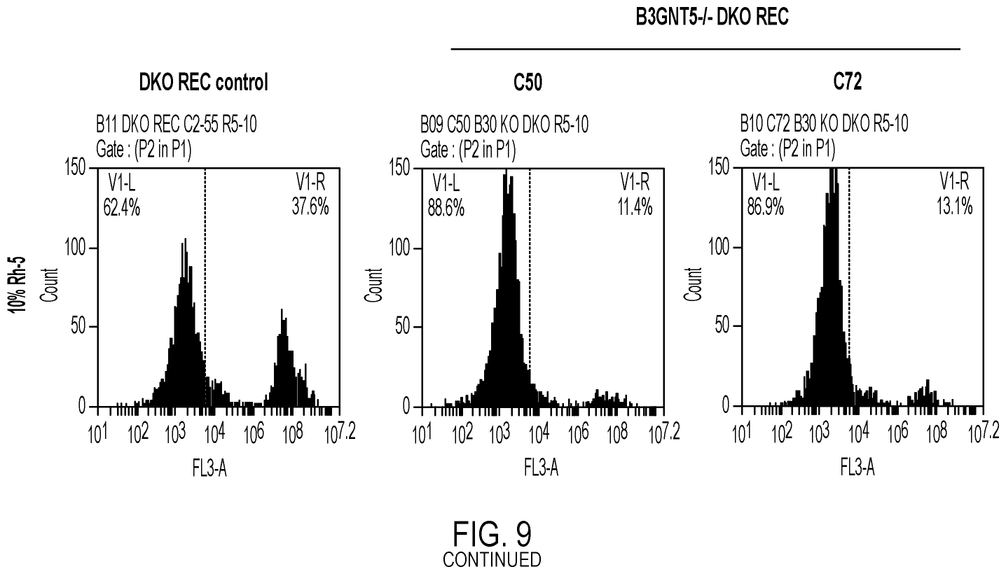

CDC assay for B3GNT5^{-/-} deficient cells following Rhesus serum treatment was performed. Incubation of B3GNT5^{-/-} sorted engineered cells with 10% serum showed less cytotoxic lysis based on a reduced amount of antibody binding (FIG. 9).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A genetically modified porcine cell comprising a reduced level of β1,3-N-acetylglucosaminyltransferase 5 activity as compared to a normal porcine cell.

2. A genetically modified porcine cell comprising a biologically inactive or deleted β1,3-N-acetylglucosaminyltransferase 5 (B3gnt5) gene.

3. The genetically modified porcine cell of claim 1, wherein the cell comprises a modification to the genome of the porcine cell that results in the lack of expression of functional β1,3-N-acetylglucosaminyltransferase 5, and wherein the genetically modified porcine cell exhibits reduced binding to human immunoglobulins relative to a porcine cell lacking the genetic modifications.

4. A genetically modified porcine cell comprising a reduced level of Lacto-N-neotetraose (LNnT) glycan (Galb1-4GlcNAcb1-3Galb1) on its cell surface as compared to a normal porcine cell.

5. The genetically modified porcine cell of claim 1, further comprising a reduced level of GlcNAc-Man3 (Mana1-6(GlcNAcb1-2Mana1-3)Manb1-4GlcNAcb) antigen.

6. The genetically modified porcine cell of claim 1, further comprising a reduced level of Sd^{a} antigen.

7. The genetically modified porcine cell of claim 1, wherein the porcine cell is selected from skin, heart, liver, kidneys, lung, pancreas, thyroid, and small bowel, or portions thereof.

8. Porcine organ or tissue comprising the genetically modified porcine cell of claim 1.

9. The porcine organ or tissue of claim 8, wherein the porcine organ or tissue is selected from skin, heart, liver, kidneys, lung, pancreas, thyroid, and small bowel, or portions thereof.

10. A method of treating a subject in need of an organ transplant, the method comprising administering a therapeutically sufficient amount of the genetically modified porcine cell of claim 1 to the subject.

11. A method of improving a rejection related symptom in a human subject comprising transplanting
(a) porcine transplant material having reduced levels of β1,3-N-acetylglucosaminyltransferase 5 activity;
(b) porcine transplant material having reduced levels of Lacto-N-neotetraose (LNnT) glycan (Galb1-4GlcNAcb1-3Galb1) on cell surfaces; or
(c) porcine transplant material having reduced levels of Lacto-N-neotetraose (LNnT) epitope (Galb1-4GlcNAcb1-3Galb1);
into a human subject in need of a transplant, wherein a rejection related symptom is improved as compared to when porcine transplant material from a wild-type pig is transplanted into a human subject.

12-13. (canceled)

14. The method of claim 11, wherein the rejection related symptom is selected from a cellular rejection response related symptom, a humoral rejection response related symptom, a hyperacute rejection related symptom, an acute humoral xenograft reaction rejection related symptom, and an acute vascular rejection response related symptom.

15. The genetically modified porcine cell of claim 2, further comprising a reduced level of GlcNAc-Man3 (Mana1-6(GlcNAcb1-2Mana1-3)Manb1-4GlcNAcb) antigen.

16. The genetically modified porcine cell of claim 2, further comprising a reduced level of Sd^{a} antigen.

17. The genetically modified porcine cell of claim 2, wherein the porcine cell is selected from skin, heart, liver, kidneys, lung, pancreas, thyroid, and small bowel, or portions thereof.

18. Porcine organ or tissue comprising the genetically modified porcine cell of claim 2.

19. The porcine organ or tissue of claim 18, wherein the porcine organ or tissue is selected from skin, heart, liver, kidneys, lung, pancreas, thyroid, and small bowel, or portions thereof.

20. A method of treating a subject in need of an organ transplant, the method comprising administering a therapeutically sufficient amount of the genetically modified porcine cell of claim 2 to the subject.

21. The genetically modified porcine cell of claim 4, further comprising a reduced level of GlcNAc-Man3 (Mana1-6(GlcNAcb1-2Mana1-3)Manb1-4GlcNAcb) antigen.

22. The genetically modified porcine cell of claim 4, further comprising a reduced level of Sd^{a} antigen.

23. The genetically modified porcine cell of claim 4, wherein the porcine cell is selected from skin, heart, liver, kidneys, lung, pancreas, thyroid, and small bowel, or portions thereof.

24. Porcine organ or tissue comprising the genetically modified porcine cell of claim 4.

25. The porcine organ or tissue of claim 24, wherein the porcine organ or tissue is selected from skin, heart, liver, kidneys, lung, pancreas, thyroid, and small bowel, or portions thereof.

26. A method of treating a subject in need of an organ transplant, the method comprising administering a therapeutically sufficient amount of the genetically modified porcine cell of claim 4 to the subject.
